(19) United States
(12) Patent Application Publication
(10) Pub. No.: US 2023/0364104 A1
(43) Pub. Date: Nov. 16, 2023

(54) PHARMACEUTICAL COMBINATION FOR THE TREATMENT OF HUMAN HYPOCHOLINERGIC DISORDERS

(71) Applicant: Kathleen E. CLARENCE-SMITH, Washington, DC (US)

(72) Inventor: Kathleen E. CLARENCE-SMITH, Washington, DC (US)

(21) Appl. No.: 18/027,020

(22) PCT Filed: Sep. 22, 2021
(86) PCT No.: PCT/US2021/051436

§ 371 Date: Mar. 17, 2023

Related U.S. Application Data
(60) Provisional application No. 63/081,643, filed on Sep. 22, 2020.
(60) Provisional application No. 63/120,503, filed on Dec. 2, 2020.
(60) Provisional application No. 63/153,488, filed on Feb. 25, 2021.
(60) Provisional application No. 63/234,290, filed on Aug. 18, 2021.

(51) Int. Cl. A61K 31/5513; A61K 31/445

(52) U.S. Cl. CPC A61K 31/5513; A61K 31/445

(57) ABSTRACT

The present invention proposes an improvement of the efficacy of donepezil for the palliative treatment of Dementia of the Alzheimer-type by counteracting its peripheral dose-limiting adverse effects with a selective peripheral M1-antagonist such as, preferably, pirenzepine, thus enabling a safe increase of the donepezil dose and consequently improved efficacy. In particular, an aim of the present invention is at least the slowing of the progression of dementia in patients suffering from a Hypocholinergic Disorder such as Alzheimer disease, Lewy body disease, Parkinson's Disease or Mild Cognitive Impairment by a safe administration of donepezil increased doses in combination with pirenzepine, as a peripheral antimuscarinic agent. This combination allows said safe treatment where other donepezil combinations failed.

### FIELD OF THE INVENTION

The present invention pertains to the field of the treatment of Hypocholinergic Disorders affecting the human central nervous system, a group of diseases that includes, but is not limited to, Dementias of the Alzheimer-type.

### OBJECT OF THE INVENTION

The present invention proposes an improvement of the efficacy of donepezil for the palliative treatment of Dementia of the Alzheimer-type by counteracting its peripheral dose-limiting adverse effects with a selective peripheral M1-antagonist such as, preferably, pirenzepine, thus enabling a safe increase of the donepezil dose and consequently improved efficacy.

In particular, the aim of the present invention is at least the slowing of the progression of dementia in patients suffering from a Hypocholinergic Disorder such as Alzheimer disease, Lewy body disease, Parkinson's Disease or Mild Cognitive Impairment by a safe administration of donepezil increased doses in combination with pirenzepine, as a peripheral antimuscarinic agent. This combination allows said safe treatment where other donepezil combinations failed.

The present invention also proposes the following embodiments
1. A selective peripheral M1 receptor antagonist selected from the group consisting of pirenzepine and pharmaceutically acceptable salts or solvates thereof for use for the treatment of Hypocholinergic Disorders in combination with a donepezil or a pharmaceutical acceptable salt or solvate thereof daily dose equivalent to from 5 mg to 80 mg of donepezil hydrochloride.
2. The selective peripheral M1 receptor antagonist for use according to embodiment 1, wherein said donepezil or pharmaceutically acceptable salt thereof is administered at daily dose equivalent to from 5 mg to 60 mg of donepezil hydrochloride.
3. The selective peripheral M1 receptor antagonist for use according to embodiment 1, wherein said donepezil or pharmaceutically acceptable salt thereof daily dose is equivalent to from 15 mg to 80 mg of donepezil hydrochloride.
4. The selective peripheral M1 receptor antagonist for use according to embodiment 1, wherein said donepezil or pharmaceutically acceptable salt thereof daily dose is equivalent from 23.01 mg to 80 mg or from 25 mg to 80 mg of donepezil hydrochloride.
5. The selective peripheral M1 receptor antagonist for use according to embodiment 1, wherein said pirenzepine and said donepezil are each formulated in a pharmaceutical composition comprising pirenzepine or a pharmaceutically acceptable salt or solvate thereof, in admixture with a pharmaceutical carrier and, respectively, donepezil or a pharmaceutically acceptable salt thereof, in admixture with a pharmaceutical carrier.
6. The selective peripheral M1 receptor antagonist for use according to embodiment 5 wherein
   said pirenzepine or pharmaceutically acceptable salt or solvate thereof is formulated in a pharmaceutical composition in an amount equivalent to from 25 mg to 600 mg of pirenzepine dihydrochloride, in admixture with a pharmaceutical carrier; and, respectively,
   said donepezil or a pharmaceutical acceptable salt thereof is also formulated in a pharmaceutical composition in an amount equivalent to from 5 mg to 80 mg of donepezil hydrochloride, in admixture with a pharmaceutical carrier.
7. The selective peripheral M1 receptor antagonist for use according to embodiment 6, wherein said donepezil or pharmaceutical acceptable salt thereof is present in said composition in an amount equivalent to from 15 mg to 80 mg of donepezil hydrochloride.
8. The selective peripheral M1 receptor antagonist for use according to embodiment 6, wherein, in said composition, said donepezil or pharmaceutical acceptable salt thereof is present in an amount equivalent to from 23.01 mg to 80 mg of donepezil hydrochloride.
9. The selective peripheral M1 receptor antagonist for use according to embodiment 5, wherein
   said pirenzepine or pharmaceutically acceptable salt or solvate thereof is formulated in a pharmaceutical composition in an amount equivalent to from 75 mg to 300 mg of pirenzepine dihydrochloride, in admixture with a pharmaceutical carrier; and, respectively,
   said donepezil or a pharmaceutical acceptable salt thereof is formulated in a pharmaceutical composition in an amount equivalent to from 25 mg to 80 mg of donepezil hydrochloride, in admixture with a pharmaceutical carrier.
10. The selective peripheral M1 receptor antagonist for use according to any one of embodiments 5 to 9, wherein said compositions are in dosage unit form and said amounts of said pirenzepine or pharmaceutical acceptable salt or solvate thereof and of said donepezil or pharmaceutical acceptable salt thereof are per unit form.
11. The selective peripheral M1 receptor antagonist for use according to embodiment 1, wherein said pirenzepine and said donepezil are both formulated in a pharmaceutical composition comprising a pharmaceutical carrier and a fixed-dose combination of said pirenzepine or a pharmaceutically acceptable salt or solvate thereof, and of said donepezil or a pharmaceutically acceptable salt thereof.
12. The selective peripheral M1 receptor antagonist for use according to any one of embodiments 1 to 11, wherein said hypocholinergic disorder is Alzheimer disease.
13. An anti-hypocholinergic disorder combination comprising
   a selective peripheral M1 receptor antagonist selected from the group consisting of pirenzepine and pharmaceutically acceptable salts and solvates thereof; and
   donepezil or a pharmaceutically acceptable salt thereof.
14. Use of a selective peripheral M1 receptor antagonist selected from the group consisting of pirenzepine and pharmaceutically acceptable salts and solvates thereof for the preparation of a medicament for the treatment of a hypocholinergic disorder in combination with donepezil or a pharmaceutically acceptable salt thereof.
15. A method for the treatment of a hypocholinergic disorder, which comprises administering to a patient in need of said treatment a selective peripheral

M1 receptor antagonist selected from the group consisting of pirenzepine and pharmaceutically acceptable salts and solvates thereof in combination with donepezil or a pharmaceutically acceptable salt thereof.
16. A pharmaceutical composition comprising
   a selective peripheral M1 receptor antagonist selected from the group consisting of pirenzepine and pharmaceutically acceptable salts and solvates thereof; and
   donepezil or a pharmaceutically acceptable salt thereof;
   in admixture with a pharmaceutical carrier.
17. The pharmaceutical composition according to embodiment 16, wherein
   said pirenzepine or pharmaceutically acceptable salt or solvate thereof is present in an amount equivalent to from 25 mg to 600 mg of pirenzepine dihydrochloride; and
   said donepezil or a pharmaceutically acceptable salt thereof is present in an amount equivalent to from 5 mg to 80 mg donepezil hydrochloride.
18. The pharmaceutical composition according to embodiment 17, wherein said donepezil or a pharmaceutically acceptable salt thereof is present in an amount equivalent to from 15 mg to 80 mg of donepezil hydrochloride.
19. The pharmaceutical composition according to embodiment 17, wherein said donepezil or a pharmaceutically acceptable salt thereof is present in an amount equivalent to from 23.01 mg to 80 mg of donepezil hydrochloride.
20. The pharmaceutical composition according to embodiment 17, wherein said pirenzepine or pharmaceutically acceptable salt or solvate thereof is present in an amount equivalent to from 75 mg to 300 mg of pirenzepine dihydrochloride; and said donepezil or pharmaceutically acceptable salt thereof is present in an amount equivalent to from 25 mg to 80 mg of donepezil hydrochloride.
21. The composition according to any one of embodiments 17 to 20, wherein said composition is in dosage unit form and said amounts of said pirenzepine or pharmaceutical acceptable salt or solvate thereof and of said donepezil or pharmaceutical acceptable salt thereof are per unit form.

### DEFINITIONS

“Dementia of the Alzheimer-type”: Dementias and other cognitive impairment resulting from decreased brain concentrations of acetylcholine associated with a loss or reduction of cholinergic neurons and/or cholinergic function, including but not limited to dementias of the Alzheimer-type such as Alzheimer's disease, Lewy Body Dementia, Parkinson's disease dementia, Mild Cognitive Impairment.

“Hypocholinergic Disorders” or “Cholinergic Deficiency Syndrome” cognitive impairment or dementia of the Alzheimer-type resulting from decreased brain concentrations of acetylcholine associated with a loss or reduction of cholinergic neurons and/or cholinergic function, including but not limited to Alzheimer's disease, Lewy Body Dementia, Parkinson's disease dementia, Mild Cognitive Impairment.

“Anti-Alzheimer”: a self-explanatory adjective standing for “adapted to the treatment of Alzheimer disease” or “for the treatment of Alzheimer disease”.

“AChE”: Acetyl Choline Esterase.

“AChEI(s)”: Acetyl Choline Esterase Inhibitor(s).

“AE”: Adverse Effect

“BBB”: Blood Brain Barrier.

“sPAChA”: selective peripheral anticholinergic agent.

“Pirenzepine”: this term, as used herein, includes 11-[(4-methylpiperazin-1-yl)acetyl]-5,11-dihydro-6H-pyrido[2,3-b][1,4]benzodiazepin-6-one and pharmaceutically acceptable salts and solvates thereof (in particular its dihydrochloride hydrate, molecular weight 442.33), unless otherwise specified. The “pirenzepine” amounts and doses are referred to pirenzepine dihydrochloride (molecular weight 424.33), unless otherwise specified.

“Donepezil”: this term, as used herein, includes (±)-2,3-dihydro-5,6-dimethoxy-2-[[1-(phenylmethyl)-4-piperidinyl]methyl]-1H-inden-1-one and pharmaceutically acceptable salts thereof, unless otherwise specified. The “donepezil” amounts and doses are referred to donepezil hydrochloride (molecular weight 415.96), unless otherwise specified.

“Equivalent to”: this expression, as used herein for any “pirenzepine or a pharmaceutically acceptable salt or solvate thereof” dose or amount and, respectively, for any “donepezil or a pharmaceutically acceptable salt thereof” dose or amount, refers to doses or amounts of said pirenzepine salts or solvates stoichiometrically equivalent to pirenzepine dihydrochloride doses or amounts (also briefly referred to as “in pirenzepine dihydrochloride”) and, respectively, to doses or amounts of said donepezil salts stoichiometrically equivalent to donepezil hydrochloride doses or amounts (also briefly referred to as “in donepezil hydrochloride”).

“Peripheral”: this term, referred to sPAChA, also applies to the selective peripheral muscarinic M1-antagonist pirenzepine that is largely unable (has a limited ability) to enter the central nervous system following oral administration and thus does not affect brain function to a clinically appreciable degree.

“MTD”: maximum (or maximal) tolerated dose, i.e. the highest dose of a drug or treatment that does not cause unacceptable side effects as determined in clinical trials by testing increasing doses on different groups of people until the highest dose with acceptable side effects is found (NCI Drug Dictionary).

“CNS”: Central Nervous System.

“PNS”: Peripheral Nervous System.

“IR”: Immediate Release of the active ingredient from a composition.

“ER”: Extended Release (or sustained release) of the active ingredient from a composition by any administration route.

### BACKGROUND OF THE INVENTION

Acetyl cholinesterase inhibitors (AChEIs) remain the therapeutic mainstay for the cognitive impairment of patients with a Hypocholinergic Disorder such as a dementia of the Alzheimer-type, despite their small effect size, dose-limiting adverse events (AEs), and the need for prolonged dose titration. PET imaging studies indicate that AChEIs given at their maximum tolerable dose (MTD) inhibit the target enzyme in brain by only about 30% (Ota et al, 2010; Kaasinen et al. 2002, Shiraishi et al, 2005), the contents of which are incorporated herein in their entirety by reference, while animal model and clinical trial results suggest that higher AChEI doses confer proportionately greater cognitive benefit. While the therapeutic benefit of AChEIs in dementia of the Alzheimer-type involves an increase of cholinergic transmission in the brain, the dose-limiting side effects involve an increase in the cholinergic transmission in the periphery.

U.S. Pat. No. 5,837,724, the contents of which are incorporated herein in their entirety by reference, discloses a method for enhancing cognition comprising administration of the anticholinergic darifenacin, chemically 2-[1-[2-(2,3-dihydrobenzofuran-5-yl)ethyl]-pyrrolidin-3-yl]-2,2-diphenyl-acetamide, and also discloses compositions comprising darifenacin and an AChEI, whereby the use of an AChEI in combination therapy may be particularly beneficial, and may have a synergistic effect. However, darifenacin is a selective M3 antimuscarinic agent (C. R Chapple et al. Expert Opin Investig Drugs. 2004 November;13(11):1493-500), the contents of which are incorporated herein in their entirety by reference, such that the alleviation of the cholinergic side effects of AChEIs must be only partial by virtue of its limited ability to inhibit gastrointestinal effects of stimulating M1 receptors located outside the CNS. In addition, only a comparison of darifenacin with oxybutynin (without an AChEI) on patients with urge incontinence is provided by this document.

Similarly, pharmacological data by Pieper et al. (DE 19612504 A1, 1997, the contents of which are incorporated herein in their entirety by reference), suggested that the adverse effects of muscarinic agonists, in particular of talsaclidine, may be alleviated by a combination with a peripheral muscarinic antagonist such as pirenzepine, ipratropium bromide, oxitropium bromide, N-butylscopolaminium bromide, trospium chloride, methantheline bromide or thiotropium bromide, with an improvement in the treatment of Alzheimer disease, in particular as far as tolerability and safety were concerned.

A benefit of alleviating the side effects of an AChEI was described in a report of four patients in whom the treatment of dementia of the Alzheimer-type with the AChEI tacrine was complicated by peripheral cholinergic gastrointestinal side effects, especially cramping, nausea, vomiting and diarrhea (Faber et al. Am J Psychiatry 156:1, 1999, page 156—“Faber 1999”, the contents of which are incorporated herein in their entirety by reference). These adverse events were ameliorated by the adjunctive use of the anticholinergic propantheline (Pro-Banthine®) at 7.5 to 15 mg taken four times a day. Based on these results, the authors recommended adjunctive use of propantheline in patients with untoward gastrointestinal cholinergic effects from cholinesterase inhibitors.

However, the authors do not suggest that antagonizing the side effects of tacrine could allow to increase the dose and the efficacy of tacrine.

Nevertheless, the aforementioned application of the general concept for improving the treatment of dementias of the Alzheimer-type provides only limited benefit to patients suffering from these disorders.

After the Faber 1999 publication, other studies concerning the safety and the BBB penetration capability of anticholinergic agents have been published.

For example, in an overview of the bladder dysfunction of dementia and Alzheimer's disease subjects, Schultz-Lampel, Urologe (A), 2003, 42, 1579-1587, the contents of which are incorporated herein in their entirety by reference, illustrate the rationale of the diagnostic and the possibilities of therapy. Among other possible drug classes, the Author cites oxybutynin, propiverine, tolterodine and trospium chloride, the last one being capable of avoiding CNS complications. No AChEI is cited in this paper.

Scheife and Takeda, Clinical Therapeutics, 2005, 27(2), 144-153, the contents of which are incorporated herein in their entirety by reference, describe the CNS adverse effects of anticholinergic drugs used for the treatment of the overactive bladder in the elderly and concludes that, when considering treatment choices for patient with overactive bladder, particularly the elderly, the potential CNS adverse effects of each anticholinergic agent must be weighed against the severity of the overactive bladder symptoms.

Siegler et al., Clin Parmacol Ther 2004; 75, 484-488 (“Siegler et al. 2004”), the contents of which are incorporated herein in their entirety by reference, studied the treatment of urinary incontinence with anticholinergics in patients taking cholinesterase inhibitors for dementia. The Authors conclude that it may be appropriate to prescribe anticholinergics and cholinesterase inhibitors together for patients with dementia who are troubled by the effects of detrusor instability and that the combination is an imperfect but often effective means of site-directed therapy in the absence of truly organ-specific medications.

WO 2004/069246, the contents of which are incorporated herein in their entirety by reference, discloses pharmaceutical compositions comprising AChEIs and anticholinergic muscarinic receptor blocking agents which cannot cross the blood-brain-barrier, said compositions being able to reduce gastro-intestinal side effects without reducing the treatment in senile dementia, thus widening the use of AChEIs for treating senile dementia. This document cites, as anticholinergic drugs, propantheline bromide, scopolamine methylbromide, isopropamide iodide, valethamate, scopolamine methobromide and methonitrate, atropine methonitrate, diponium bromide, pipenzolate bromide, penthienate bromide, benactizine methobromide and dibutoline sulfate. However, the specifically described compositions are all made with propantheline bromide as anticholinergic agent. In particular, the cited document neither discloses nor suggests that certain anticholinergic drugs may improve the treatment of Hypocholinergic Disorders not only in terms of lessening the side effects of AChEIs but also in terms of enabling a safe increase in the dose of AChEIs and thus an increase in the efficacy on the symptoms of dementia.

In a series of documents, A. K. Gunnar Aberg discloses the use of the anticholinergic trospium for treating urinary incontinence (US 2005/0043342, now U.S. Pat. No. 6,974,820), smooth muscle disorders in patients suffering from cardiac contractility disorders (US 2007/0004766) and smooth muscle disorders patients suffering from memory disorders (US 2006/0293356), the contents of which are incorporated herein in their entirety by reference. According to this last document, smooth muscle disorders in patients suffering from a memory disorder may be treated with trospium while avoiding drug-induced memory disorders or drug-induced worsening of existing memory disorders. Even though this document states that the AChEIs have opposite effects to those of common anticholinergic drugs, it neither mentions nor suggests the possibility of improving the symptoms of Hypocholinergic disorders by combining an AChEI and trospium.

In a pharmacological study in mice using oral tolterodine, Cappon et al., Eur. J. Pharmacol., 2008, 579, 225-228, the contents of which are incorporated herein in their entirety by reference, observed that the tested drug had no effect on memory in the mouse passive avoidance model and concluded that tolterodine does not disrupt cognitive function under the test conditions. No AChEI is cited in this document.

These observations led the present inventors to hypothesize that, if the dose-limiting side effects of AChEI could be attenuated by blocking the increased stimulation of muscarinic cholinergic receptors in the periphery, higher AChEI doses could be safely administered, and greater efficacy would be obtained.

In fact, an improvement in the treatment of the cognitive impairment of dementia of the Alzheimer-type was attained by a combined therapy associating a non-selective, peripheral anticholinergic agent, at a dose of from 20% to 200% the current daily doses, with an AChEI, at a dose up to about 4 times the maximal recommended dose of said AChEI, as disclosed in U.S. Pat. No. 8,404,701, the contents of which are incorporated herein in their entirety by reference. By such a treatment, a higher acetylcholinesterase inhibition in the CNS is achieved and greater relief of the symptoms of Dementia of the Alzheimer-type is enabled, by concomitantly decreasing concurrent adverse effects.

In addition, U.S. Pat. No. 8,877,768, the contents of which are incorporated herein in their entirety, discloses an improvement in the treatment of Dementia of the Alzheimer-type which is attained by a combined therapy associating a non-anticholinergic antiemetic agent, at a dose of from 50% to 300% the current IR daily doses, with an AChEI, at a dose up to 4 times the maximal recommended doses of said AChEI when administered alone.

Similarly, WO 2014/039627, the contents of which are incorporated herein in their entirety, discloses the discovery of the property of the non-selective, peripheral anticholinergic agent of increasing the blood levels of a concurrently administered AChEI, the higher being the dose of either the non-selective anticholinergic agent or the AChEI, the higher being the increase of the AChEI blood levels. Thus, this document recommends the use of high doses of both the non-selective, peripheral anticholinergic agent and of the AChEI in order to ameliorate the symptoms of dementia of the Alzheimer-type. In particular, this document states that “[W]while potentially lessening side effects and thereby enabling the use of higher and thus more effective doses of the AChEI, merely employing the concomitant use of antiemetics, such as domperidone and others, or of anticholinergics such as propantheline, oxybutynin, tolterodine and others, falls short of achieving the utmost therapeutic advantages of AChEIs in the treatment Alzheimer-type dementias”.

Thus, U.S. Pat. No. 8,404,701 and, especially, WO 2014/039637 specifically exclude anticholinergic agents which are selective and/or non-peripheral because selective agents are not able to counteract the whole spectrum of the AChEIs' adverse effect and, worse, the non-peripheral anticholinergics, such as oxybutynin, are able to dangerously counteract the beneficial central action of said AChEIs.

Clinical trials confirmed the hypothesis of the possibility of increasing the donepezil doses. In a Phase I single-blind, placebo-controlled, cross-over, dose-escalation study of oral donepezil to MTD or to protocol limit, co-administration of solifenacin, a non-selective peripheral muscarinic receptor antagonist, attenuated dose-limiting AEs of donepezil and enabled the safe and tolerable administration of higher donepezil doses up to 40 mg/day (Chase and Clarence-Smith, AAIC Abstract #2729l, 2015), the contents of which are incorporated herein in their entirety by reference.

Following this study in healthy volunteers, a Phase 2a study of the combination donepezil-solifenacin was conducted in patients with Alzheimer's disease (Chase et al. Neurotherapeutics 14:405-416, 2017). The primary outcome measure was the maximum tolerated dose (MTD) of donepezil achieved (to protocol limit of 40 mg/day), when administered with the anticholinergic solifenacin 15 mg/day (note that the approved doses for solifenacin are 5 and 10 mg/day). Secondary measures included assessments of cognitive and global function, as well as of AEs. The mean±SD donepezil MTD increased to 38±0.74 mg/day; p<0.001); 88% of the study population safely attained this dose at the end of titration. Markedly reduced donepezil AE frequency, especially gastrointestinal, allowed this dose increase. At 26 weeks, Alzheimer's Disease Assessment Scale Cognitive Component (ADAS-Cog) scores in the efficacy evaluable population improved by 0.35±0.85 points over baseline (p<0.05), the Clinical Global Impression (CGI) of Improvement scores improved by 0.94±0.20 to 3.1±0.20 points (p<0.001). The findings show that limiting donepezil AEs by co-administration of solifenacin allows the safe administration of substantially higher donepezil doses that provide significantly increased anti-dementia efficacy. The findings also suggest that higher donepezil doses might have provided greater antidementia efficacy.

Unfortunately, in humans solifenacin causes an increase in QTc (Asajima et al 2008; Newgreen et al, 2017; and Heranval et al, 2016, the contents of which are incorporated herein in their entirety by reference), thus limiting the safety of the combination with donepezil.

Further research, using in vitro myocardial tissue on the role of M1, M2, and M3 muscarinic receptor antagonists on the regulation of electrical activity of the myocardium, showed that: acetylcholine (1 μM) suppressed automatic activity in the myocardium, perfusion of the preparation with non-selective blocker atropine (1 μM) completely abolished the effect of acetylcholine, treatment with the M2 receptor blocker AQ-RA 741 (i.e. 11-[[4-[4-(diethylamino)butyl]-1-piperidinyl]acetyl]-5,11-dihydro-6H-pyrido[2,3-b] [1,4]benzodiazepin-6-one, 1 μM) led to a partial suppression of the effect of acetylcholine. Blockers of M1 and M3 receptors pirenzepine (1 μM) and 4DAMP (i.e, 4-[(Diphenylacetyl)oxy]-1,1-dimethylpiperidin-1-ium iodide, 0.1 μM) did not suppress the effect of acetylcholine. Thus, the effect of acetylcholine on the heart is predominantly realized via M2 receptors (Ivanova AD, Tapilina SV, Kuz'min VS. Role of Muscarinic M1, M2, and M3 Receptors in the Regulation of Electrical Activity of Myocardial Tissue of Caval Veins during the Early Postnatal Ontogeny. Bull Exp Biol Med. 2019 February;166(4):421-425. doi: 10.1007/s10517-019-04364-9. Epub 2019 Feb. 19. PMID: 30783837).

Similarly, most peripheral anticholinergic drugs act on several muscarinic receptors and not only on the M1 receptor. Yet, blockade of the M1 receptor underlies the dose-limiting side effects of donepezil. Consequently, the use of anticholinergic drugs to enable the increase in the dose of donepezil and therefore its efficacy in the treatment of hypocholinergic disorders carries the risk of causing additional adverse events through their effects on muscarinic receptors other than the M1 receptor.

US 2010/0247688, the contents of which are incorporated herein in their entirety by reference, discloses the use of pirenzepine as an anti-cerebral amyloidosis drug for slowing or stopping the progression of neuronal degeneration that underlies Alzheimer's disease. US 2010/0247688 also mentions that pirenzepine antagonizes the dose-limiting adverse effects of AChEIs, leading to greater comfort for the patients.

However, even though US 2010/0247688 discloses the use of pirenzepine for the treatment of a cerebral disease, it is well known that pirenzepine does not cross the BBB, as described by Eberlein et al. in Arzneimittel Forschung 27,356-359 (1977), the contents of which are incorporated herein in their entirety by reference, and also confirmed by the whole literature that followed, thus rendering any action of pirenzepine in the CNS impossible. In fact, the same first inventor of the above US 2010/0247688 correctly admitted the inactivity of pirenzepine in the treatment of AD in a 31 Jan. 2013 press release.

In summary, there was and there continues to be an urgent need to safely enhance the dose regimen of donepezil in order to allow a substantive improvement in the symptoms of dementia of the Alzheimer-type and, by consequence, the problem of safely increasing the donepezil dose for this purpose remains unresolved.

### SUMMARY OF THE INVENTION

Unlike the teachings of U.S. Pat. No. 8,404,701 (that proposed blocking all the peripheral muscarinic receptors) and of US2010/0247688 (that proposes the use of pirenzepine within the BBB in order to directly treat amyloidosis), the present invention provides the use of orally administered pirenzepine for selectively inhibiting the activation of M1 muscarinic receptors in the PNS, but not in the CNS. Such an approach allows the mitigation of the GI dose limiting adverse events of donepezil, such that higher doses of donepezil can be safely administered leading to greater and more prolonged antidementia efficacy with fewer peripheral GI dose-limiting adverse events. The use of a selective M1 muscarinic receptor antagonist is justified since most dose-limiting adverse effects with donepezil are GI adverse effects, shown to be mediated by the M1 muscarinic receptor (Alt et al, Evidence for Classical Cholinergic Toxicity Associated with Selective Activation of M1 Muscarinic Receptors, 2016). Inhibiting other muscarinic receptors could lead to adverse effects related to the inhibitor (for example, cardiovascular effects with solifenacin; Asajima et al 2008; Newgreen et al, 2017; Heranval et al, 2016).

In particular, a selective antagonist of peripheral M1-muscarinic receptors can attenuate dose-limiting adverse effects of donepezil without the risk of “new” adverse events such as prolongation of QTc. More particularly, pirenzepine is a selective antagonist of the M1 receptor and thus appears to be uniquely suited to the safe enablement of high doses of donepezil causing a significant increase in antidementia efficacy and improved cognition.

Thus, it is a first aspect of the present invention to provide a method for increasing the maximal tolerated dose of donepezil in a patient suffering from a Hypocholinergic Disorder such as dementia of the Alzheimer-type without concurrent, appreciable adverse effects. This method comprises administering to said patient a selective peripheral M1 receptor antagonist, preferably pirenzepine or a pharmaceutically acceptable salt or solvate thereof, in combination with a donepezil daily dose (in donepezil hydrochloride) of from 5 mg to 80 mg, whereby an enhanced acetyl choline esterase inhibition in the CNS of said patient is achieved and the symptoms of a Hypocholinergic Disorder such as a dementia of the Alzheimer-type in said patient are improved. In this treatment, the donepezil daily doses (in donepezil hydrochloride) include low doses used during the titration period.

According to an embodiment, said donepezil daily dose is equivalent to from 5 mg to 60 mg of donepezil hydrochloride.

A second aspect of the invention provides for the use of a selective peripheral M1 receptor antagonist, preferably pirenzepine or a pharmaceutically acceptable salt or solvate thereof, for the preparation of a medicament, normally consisting of a pharmaceutical composition for the treatment of a Hypocholinergic Disorder such as dementia of the Alzheimer-type in combination with donepezil or a pharmaceutically acceptable salt thereof also in a pharmaceutical composition comprising said donepezil or pharmaceutically acceptable salt thereof in an amount equivalent to from 5 mg to 80 mg of donepezil hydrochloride.

According to an embodiment, said donepezil in said or pharmaceutically acceptable salt thereof is present in said composition in an amount equivalent to from 5 mg to 60 mg of donepezil hydrochloride.

A third aspect of the invention provides a selective peripheral M1 receptor antagonist, preferably pirenzepine or a pharmaceutically acceptable salt or solvate thereof, for use in the treatment of a Hypocholinergic Disorder such as dementia of the Alzheimer-type in a patient, in combination with a donepezil or a pharmaceutical acceptable salt thereof, at a daily dose equivalent to from 5 mg to 80 mg of donepezil hydrochloride.

According to an embodiment, said donepezil daily dose is equivalent to from 5 mg to 60 mg of donepezil hydrochloride.

A fourth aspect of the present invention provides a pharmaceutical anti-hypocholinergic disorder combination, comprising a selective peripheral M1 receptor antagonist, preferably pirenzepine, and a donepezil dose (in donepezil hydrochloride) of from 5 mg to 80 mg.

According to an embodiment, said donepezil daily dose is equivalent to from 5 mg to 60 mg of donepezil hydrochloride.

In particular, this fourth aspect of the invention provides a pharmaceutical anti-Alzheimer combination comprising pirenzepine or a pharmaceutically acceptable salt or solvate thereof and donepezil or a pharmaceutical acceptable salt thereof daily dose equivalent to from 5 mg to 80 mg of donepezil hydrochloride, including daily doses used in the titration period, for combating dementia in a patent suffering from a Hypocholinergic Disorder such as Alzheimer's disease, Parkinson's disease, Lewy body disease or Mild Cognitive Impairment.

Thus, by the increase of the maximal tolerated dose of donepezil, a higher degree of acetyl choline esterase inhibition in the CNS is safely achieved and the cognitive symptoms of a Hypocholinergic Disorder, such as dementia in a patient is improved to a greater extent without concurrent, appreciable adverse effects.

According to the above four aspects of the invention, pirenzepine is administered to said patient at a daily dose equivalent to from 25 mg to 600 mg of pirenzepine dihydrochloride in combination with a donepezil daily dose equivalent to from 5 mg to 80 mg of donepezil hydrochloride. Alternatively, said pirenzepine daily dose is equivalent to from 75 mg to 300 mg of pirenzepine dihydrochloride, in combination with a donepezil daily dose equivalent to from 5 mg to 80 mg of donepezil hydrochloride.

Normally, according to the above four aspects of the invention, pirenzepine or a pharmaceutically acceptable salt or solvate thereof and donepezil or a pharmaceutically acceptable salt thereof are each formulated in a pharmaceutical composition comprising, respectively, said pirenzepine or a pharmaceutically acceptable salt or solvate thereof, in an amount equivalent to from 25 mg to 600 mg of pirenzepine dihydrochloride, in admixture with pharmaceutical carrier or vehicle, and said donepezil or a pharmaceutically acceptable salt thereof, in an amount equivalent to from 5 mg to 80 mg of donepezil hydrochloride, in admixture with pharmaceutical carrier or vehicle. Alternatively, in said composition, said donepezil or pharmaceutically acceptable salt thereof is present in an amount equivalent to from 5 mg to 60 mg of donepezil hydrochloride.

According to a specific embodiment of the above four aspects of the invention, pirenzepine or a pharmaceutically acceptable salt or solvate thereof is formulated in the above composition in an amount equivalent to from 25 mg to 300 mg of pirenzepine dihydrochloride; and, respectively, said donepezil or a pharmaceutically acceptable salt thereof is formulated in the above respective composition in an amount equivalent to from 15 mg to 80 mg of donepezil hydrochloride. Alternatively, said donepezil or pharmaceutically acceptable salt thereof is present in said composition in an amount equivalent to from 15 mg to 60 mg of donepezil hydrochloride.

According to the above four aspects of the invention, pirenzepine or a pharmaceutically acceptable salt or solvate thereof and donepezil or a pharmaceutically acceptable salt thereof may also be each formulated in a pharmaceutical composition comprising, respectively,
said pirenzepine or a pharmaceutically acceptable salt or solvate thereof, in an amount equivalent to from 75 mg to 300 mg of pirenzepine dihydrochloride. in admixture with a pharmaceutical carrier or vehicle; and
said donepezil or a pharmaceutically acceptable salt thereof, in an amount equivalent to from 23.01 mg to 80 mg of donepezil hydrochloride, in admixture with a pharmaceutical carrier or vehicle. Preferably, in said composition, said donepezil is present in an amount equivalent to from 25 mg to 80 mg.

Alternatively, according to the above four aspects of the invention, pirenzepine or a pharmaceutically acceptable salt or solvate thereof and donepezil or a pharmaceutically acceptable salt thereof are each formulated in a pharmaceutical composition comprising, respectively, said pirenzepine or a pharmaceutically acceptable salt or solvate thereof, in an amount equivalent to from 75 mg to 300 mg of pirenzepine dihydrochloride, in admixture with a pharmaceutical carrier or vehicle; and said donepezil or a pharmaceutically acceptable salt thereof, in an amount equivalent to from 25 mg to 60 mg or from 25 mg to 40 mg of donepezil hydrochloride, in admixture with a pharmaceutical carrier or vehicle.

Preferably, according to the above four aspects of the invention, said pharmaceutical compositions are in dosage unit form, and the above amounts of pirenzepine or pharmaceutically acceptable salt or solvate thereof and, respectively, of donepezil or pharmaceutically acceptable salt thereof are per unit form.

In the case of separate (concurrent or sequential) administration of said pirenzepine, in an effective amount per unit form equivalent to from 25 mg to 600 mg of pirenzepine dihydrochloride, and of said donepezil, in an effective amount per unit form equivalent to from 15 mg to 80 mg of donepezil hydrochloride, each of them may be packaged in a kit comprising said pirenzepine, in admixture with a pharmaceutical carrier or vehicle, in a container; and said donepezil, in admixture with a pharmaceutical carrier or vehicle, in another, separate container. Alternatively, in said kit, donepezil hydrochloride may be packaged in an amount per unit form equivalent to from 23.01 mg to 80 mg of donepezil hydrochloride.

Preferably, in said kit, pirenzepine or pharmaceutically acceptable salt or solvate thereof is packaged in an amount per unit form equivalent to from 75 mg to 300 mg of pirenzepine dihydrochloride, and said donepezil or pharmaceutically acceptable salt thereof, is packaged in an amount per unit form equivalent to from 23.01 mg to 80 mg of donepezil hydrochloride. Alternatively, in said preferred kit, donepezil or a pharmaceutically acceptable salt thereof is packaged in an amount per unit form equivalent to from 25 mg to 80 mg of donepezil hydrochloride.

In the case of separate (concurrent or sequential) administration of said pirenzepine, in an effective amount per unit form equivalent to from 75 mg to 300 mg of pirenzepine dihydrochloride, and of said donepezil, in an effective amount per unit form equivalent to from 23.01 mg to 80 mg of donepezil hydrochloride, each of them can be packaged in a kit comprising said pirenzepine, in admixture with a pharmaceutical carrier or vehicle, in a container; and said donepezil, in admixture with a pharmaceutical carrier or vehicle, in another, separate container. In said separate container, said donepezil is preferably present, in admixture with a pharmaceutical carrier or vehicle, in an amount per unit form equivalent to from 25 mg to 80 mg of donepezil hydrochloride.

Alternatively, in the separate container of said kit containing pirenzepine, said pirenzepine is present in an amount per unit form equivalent to from 75 mg to 300 mg of pirenzepine dihydrochloride and, respectively, in the separate container of said kit containing donepezil, said donepezil is present in an amount per unit form equivalent to from 25 mg to 60 mg or from 25 mg to 40 mg of donepezil hydrochloride.

A fifth aspect of the present invention provides a pharmaceutical composition for use in the treatment of dementia in a patient suffering from a Hypocholinergic Disorder, comprising a pharmaceutical carrier or vehicle and a fixed-dose combination of pirenzepine or a pharmaceutically acceptable salt or solvate thereof, in an amount equivalent to from 25 mg to 600 mg of pirenzepine dihydrochloride, and donepezil or a pharmaceutically acceptable salt thereof, in an amount equivalent to from 5 mg to 80 mg of donepezil hydrochloride.

Alternatively, this fifth aspect of the present invention provides a pharmaceutical composition for the improvement of the cognitive symptoms of a Hypocholinergic Disorder selected from the group consisting of Alzheimer's Disease, Lewy Body Dementia, Parkinson's disease dementia, or Mild Cognitive Impairment, comprising a pharmaceutical carrier or vehicle and a fixed-dose combination of pirenzepine or a pharmaceutically acceptable salt or solvate thereof, in an amount equivalent to from 25 mg to 600 mg of pirenzepine dihydrochloride, and donepezil or a pharmaceutically acceptable salt thereof, in an amount equivalent to from 5 mg to 80 mg of donepezil hydrochloride.

In said pharmaceutical composition donepezil or a pharmaceutically acceptable salt thereof may be present in an amount equivalent to from 5 mg to 60 mg or from 7.5 mg to 60 mg or from 7.5 mg to 50 mg of donepezil hydrochloride.

According to an embodiment of this fifth aspect, the above pharmaceutical composition comprises a pharmaceutical carrier or vehicle and a fixed-dose combination of pirenzepine or a pharmaceutically acceptable salt or solvate thereof, in an amount equivalent to from 75 mg to 300 mg of pirenzepine dihydrochloride, and donepezil or a pharmaceutically acceptable salt thereof, in an amount equivalent to from 23.01 mg to 80 mg of donepezil hydrochloride. Preferably, this amount is equivalent to from 25 mg to 80 mg of donepezil hydrochloride.

Alternatively, according to this embodiment of this fifth aspect, said donepezil or pharmaceutically acceptable salt thereof may be present in said fixed-dose combination in an amount equivalent to from 25 mg to 60 mg or from 25 mg to 40 mg of donepezil hydrochloride.

A sixth aspect of the present invention provides a pharmaceutical composition comprising
pirenzepine or a pharmaceutically acceptable salt or solvate thereof, in an amount equivalent to from 25 mg to 600 mg of pirenzepine dihydrochloride; and
donepezil or a pharmaceutically acceptable salt thereof, in an amount equivalent to from 5 mg to 80 mg of donepezil hydrochloride,
in admixture with a pharmaceutical carrier or vehicle.

Alternatively, according to this sixth aspect, said donepezil or pharmaceutically acceptable salt thereof may be present in said composition in an amount equivalent to from 5 mg to 60 mg or from 7.5 mg to 50 mg of donepezil hydrochloride.

According to an embodiment of this sixth aspect, in said pharmaceutical composition said pirenzepine or pharmaceutically acceptable salt or solvate thereof is present in an amount equivalent to from 25 mg to 600 mg of pirenzepine dihydrochloride, and said donepezil or pharmaceutically acceptable salt thereof is present in an amount equivalent to from 10.01 mg to 80 mg of donepezil hydrochloride.

Alternatively, according to this embodiment of this sixth aspect, donepezil or a pharmaceutically acceptable salt thereof is present in said composition in an amount-range (in donepezil hydrochloride) selected from the group consisting from 10.01 mg to 60 mg, from 12.5 mg to 60 mg, from 12.5 mg to 50 mg, from 12.5 mg to 40 mg, and from 25 mg to 30 mg.

A preferred embodiment of this sixth aspect of the present invention provides a pharmaceutical composition comprising
pirenzepine or a pharmaceutically acceptable salt or solvate thereof, in an amount equivalent to from 75 mg to 600 mg of pirenzepine dihydrochloride; and
donepezil or a pharmaceutically acceptable salt thereof, in an amount equivalent to from 23.01 mg to 80 mg of donepezil hydrochloride,
in admixture with a pharmaceutical carrier or vehicle.

Preferably said donepezil or a pharmaceutically acceptable salt thereof is present in said composition in an amount equivalent to from 25 mg to 80 mg of donepezil hydrochloride.

Alternatively, this preferred embodiment of this sixth aspect of the present invention provides a pharmaceutical composition comprising pirenzepine or a pharmaceutically acceptable salt or solvate thereof in an amount equivalent to from 75 mg to 600 mg of pirenzepine dihydrochloride, and donepezil or a pharmaceutically acceptable salt thereof in an amount equivalent to from 25 mg to 60 mg or from 25 mg to 40 mg of donepezil hydrochloride, in admixture with a pharmaceutical carrier or vehicle.

Normally, the above pharmaceutically compositions according to these six aspects of the invention are in dosage unit form and the above amounts or amount ranges are per unit form.

In particular, the invention provides a pharmaceutical composition in dosage unit form comprising
(a) pirenzepine or a pharmaceutically acceptable salt or solvate thereof, in an amount per unit form equivalent to from 75 mg to 600 mg of pirenzepine dihydrochloride, and
(b) donepezil or a pharmaceutically acceptable salt thereof, in an amount per unit form equivalent to from 23.01 mg to 80 mg of donepezil hydrochloride, in admixture with a pharmaceutical carrier or vehicle.

More particularly, the invention provides a pharmaceutical composition in dosage unit form comprising
(a) pirenzepine or a pharmaceutically acceptable salt or solvate thereof, in an amount per unit form equivalent to from 75 mg to 300 mg of pirenzepine dihydrochloride, and
(b) donepezil or a pharmaceutically acceptable salt thereof, in an amount per unit form equivalent to from 25 mg to 80 mg of donepezil hydrochloride,
in admixture with a pharmaceutical carrier or vehicle.

According to this preferred embodiment of this sixth aspect of the present invention, these particular compositions may comprise donepezil or a pharmaceutically acceptable salt thereof in an amount per unit form equivalent to from 25 mg to 60 mg or from 25 mg to 40 mg of donepezil hydrochloride.

The compositions according to this sixth aspect of the invention are useful or for use in the treatment of a Hypocholinergic Disorder such as dementia of Alzheimer type or Alzheimer's disease. More particularly these compositions are useful for preventing or treating dementia in a patient suffering from a hypocholinergic disorder such as Alzheimer disease, Parkinson's disease, Lewy body disease or Mild Cognitive Impairment.

### DETAILED DESCRIPTION

The present invention proposes an improved method to augment the efficacy of donepezil for the palliative treatment of Hypocholinergic syndromes such as dementias of the Alzheimer-type. Donepezil's efficacy involves the stimulation of M1 cholinergic receptors in the CNS, but its dose-limiting GI adverse effects involve the stimulation of M1 receptors in the PNS. Pirenzepine acts to selectively inhibit the activation of M1 muscarinic receptors in the PNS, but not in the CNS, thereby mitigating the GI dose limiting adverse events of donepezil. Consequently, higher doses of donepezil can be safely administered leading to greater and more prolonged antidementia efficacy with fewer peripheral GI dose-limiting adverse events.

### The Pirenzepine

Pirenzepine, chemically 11-[(4-methylpiperazin-1-yl)acetyl]-5,11-dihydro-6H-pyrido[2,3-b][1,4]benzodiazepin-6-one of formula

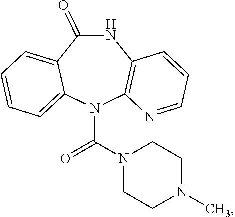 [SMILES: CN1CCN(C(=O)N2c3ccccc3C(=O)Nc3cccnc32)CC1]

is an antiulcer anticholinergic product known as from 1970 (BE 737748; see also U.S. Pat. No. 3,660,380), acting as a selective, peripheral muscarinic M1-antagonist.

Pirenzepine dihydrochloride is used, in its antiulcer indication, in tablets comprising 25 mg or 50 mg of pirenzepine dihydrochloride and is administered from once per day to three times per day.

According to the present invention, pirenzepine is used, in combination—including fixed-dose combinations—with donepezil or a pharmaceutically acceptable salt thereof, in a pharmaceutical composition comprising pirenzepine or a pharmaceutically acceptable salt or solvate thereof in an amount equivalent to from 25 mg to 600 mg of pirenzepine dihydrochloride, in admixture with a pharmaceutical carrier or vehicle. Normally, said composition is in dosage unit form.

In particular, pirenzepine may be used, in said combination—including fixed-dose combinations—with donepezil or a pharmaceutically acceptable salt thereof, in a pharmaceutical composition comprising pirenzepine or a pharmaceutically acceptable salt or solvate thereof in an amount equivalent to a range selected from the group consisting of from 50 mg to 600 mg, from 100 mg to 600, from 150 mg to 600 mg and from 150 mg to 500 mg of pirenzepine dihydrochloride.

According to a preferred embodiment, pirenzepine is in a pharmaceutical composition in dosage unit form comprising pirenzepine or a pharmaceutically acceptable salt or solvate thereof, in an amount per unit form equivalent to from 75 mg to 300 mg of pirenzepine dihydrochloride, in admixture with a pharmaceutical carrier or vehicle. Said composition may be in IR-formulation or ER-formulation.

Pirenzepine is administered, in said compositions, at a daily dose (in pirenzepine dihydrochloride) of from 25 mg to 600 mg in combination—including fixed-dose combinations—with donepezil or a pharmaceutically acceptable salt thereof, at a daily dose equivalent to from 5 mg to 80 mg of donepezil hydrochloride, said daily dose including low (5-10 mg) daily doses administered during to the titration period.

In particular, for this treatment, pirenzepine is in a pharmaceutical composition comprising pirenzepine or a pharmaceutically acceptable salt or solvate thereof, in an amount equivalent to from 75 mg to 300 mg of pirenzepine dihydrochloride, to be administered to a patient suffering from a Hypocholinergic Disorder in combination with a donepezil or a pharmaceutically acceptable salt thereof daily dose equivalent to from 23.01 mg to 80 mg of donepezil hydrochloride.

Preferably, said donepezil daily dose is equivalent to from 25 mg to 80 mg donepezil hydrochloride.

According to an embodiment, the invention provides a pharmaceutical composition for use in the treatment of a hypocholinergic disorder, said composition comprising a pharmaceutical carrier or vehicle and a fixed-dose combination of pirenzepine or a pharmaceutically acceptable salt or solvate thereof and donepezil or a pharmaceutically acceptable salt thereof.

In particular, in this pharmaceutical composition, said pirenzepine or pharmaceutically acceptable salt or solvate thereof is present in an amount equivalent to from 25 mg to 600 mg of pirenzepine hydrochloride and said donepezil or pharmaceutically acceptable salt thereof is present in an amount equivalent to from 5 mg to 80 mg of donepezil hydrochloride.

Preferably, in this pharmaceutical composition, said pirenzepine or pharmaceutically acceptable salt or solvate thereof is present in an amount equivalent to from 75 mg to 300 mg of pirenzepine hydrochloride and said donepezil or pharmaceutically acceptable salt thereof is present in an amount equivalent to from 23.01 mg to 80 mg or from 25 mg to 80 mg of donepezil hydrochloride.

According to a preferred embodiment, the invention provides a pharmaceutical composition comprising pirenzepine or a pharmaceutically acceptable salt or solvate (preferably the hydrate) thereof in an amount equivalent to from 25 mg to 600 mg of pirenzepine dihydrochloride, and of donepezil or a pharmaceutically acceptable salt or solvate thereof in an amount equivalent to from 5 mg to 60 mg of donepezil hydrochloride. This composition is in a dosage unit form and the above amounts of pirenzepine and donepezil are per unit form.

More particularly, the invention provides a pharmaceutical composition in dosage unit form, which comprises pirenzepine or a pharmaceutically acceptable salt or solvate thereof, in an amount per unit form equivalent to from 25 mg to 600 mg of pirenzepine hydrochloride; and donepezil or a pharmaceutically acceptable salt thereof, in an amount per unit form equivalent to from 5 mg to 80 mg of donepezil hydrochloride, in admixture with a pharmaceutical carrier or vehicle.

In said composition, said pirenzepine or pharmaceutically acceptable salt or solvate thereof may be present in an amount equivalent to from 25 mg to 300 mg of pirenzepine dihydrochloride in an IR-formulation, or in an amount equivalent to from 25 mg to 600 mg in ER-formulation.

According to an embodiment, in said composition,
said pirenzepine or pharmaceutically acceptable salt or solvate thereof is present
in an amount equivalent to a range selected form the group consisting of from 50 mg to 300 mg, from 75 mg to 300 mg, from 100 mg to 300 mg, from 150 mg to 300 mg and from 150 mg to 250 mg, of pirenzepine dihydrochloride in an IR-formulation; or
in an amount equivalent to a range selected from the group consisting of from 50 mg to 600 mg, from 75 mg to 300 mg, from 100 mg to 600, from 150 mg to 600 mg and from 150 mg to 500 mg of pirenzepine dihydrochloride in an ER-formulation; and
said donepezil or pharmaceutically acceptable salt thereof is present in an amount equivalent to a range selected form the group consisting of from 5 mg to 60 mg, from 10.01 mg to 60 mg, from 12.5 mg to 60 mg, from 12.5 mg to 50 mg, from 12.5 mg to 40 mg and from 25 mg to 30 mg of donepezil hydrochloride.

Preferably, according to this embodiment, said composition comprises pirenzepine or pharmaceutically acceptable salt or solvate thereof in an amount equivalent to from 75 mg to 600 mg, normally from 75 mg to 300 mg of pirenzepine dihydrochloride; and donepezil or pharmaceutically acceptable salt thereof is present in said composition in an amount equivalent to from 23.01 mg to 80 mg, normally from 25 mg to 80 mg, from 25 mg to 60 mg or from 25 mg to 40 mg, of donepezil hydrochloride.

According to a preferred embodiment, the invention provides a pharmaceutical composition comprising pirenzepine or a pharmaceutically acceptable salt or solvate thereof, in an amount equivalent to from 75 mg to 300 mg of pirenzepine dihydrochloride; and donepezil or a pharmaceutically acceptable salt thereof, in an amount equivalent to from 25 mg to 80 mg of donepezil hydrochloride, in admixture with a pharmaceutical carrier or vehicle. This preferred composition may be in IR-formulation or in ER-formulation.

This composition is especially indicated for the treatment of a Hypocholinergic Disorder after the titration period during the Maintenance Period.

As set forth above, the above compositions are in dosage unit form and the above amounts of pirenzepine and donepezil are per unit form. A specific pirenzepine solvate is the monohydrate thereof.

In conclusion, according to the present invention, pirenzepine may be indicated for the treatment of dementia in a patient suffering from a Hypocholinergic Disorder, for example of Alzheimer's disease, Parkinson's disease, Lewy body disease or Mild Cognitive Impairment in a patient under treatment with donepezil.

### The Donepezil

Donepezil, chemically (RS)-2-[(1-benzyl-4-piperidyl)methyl]-5,6-dimethoxy-2,3-dihydroinden-1-one of formula

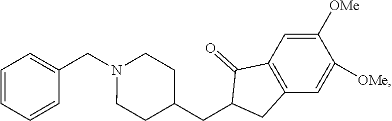

wherein Me stands for methyl, is an acetylcholinesterase inhibitor presented, as hydrochloride, in orally disintegrating tablets or in tablets to be swallowed containing 5 or 10 mg per tablet to be administered once a day. It is used for the treatment of dementia of the Alzheimer-type at recommended daily dosages of from 5 to 10 mg to be administered once a day, and as a dose formulation containing 23 mg of donepezil hydrochloride in a matrix type tablet to be administered once per day.

Currently, the 5-mg and 10-mg tablets are in IR-formulation and the 23-mg matrix-type tablets are in ER-formulation, for sustained release.

As set forth in the preceding “The pirenzepine” section, when used with or combined with pirenzepine, and thanks to the protective action of said pirenzepine, donepezil may be administered at daily doses higher, and even much higher than the presently maximum recommended dose (10 mg, or 23 mg in a matrix-type tablet). This pirenzepine protective action, unlike that of the teachings of U.S. Pat. No. 8,404,701, selectively antagonizes the donepezil adverse effects at the peripheral M1-muscarinic receptors responsible of the GI functions only, while allowing donepezil to act beneficially on, for example, the central nervous system muscarinic receptors.

According to the present invention said donepezil or pharmaceutically acceptable salts thereof is in a pharmaceutical composition comprising a pharmaceutical carrier and a donepezil amount equivalent to from 5 mg to 80 mg of donepezil hydrochloride. This composition is adapted to be administered to a patient suffering from a Hypocholinergic Disorder such as dementia of Alzheimer type at a daily dose equivalent to from 5 mg to 80 mg of donepezil hydrochloride, in combination with pirenzepine or a pharmaceutically acceptable salt or solvate thereof. For the treatment of said patient, pirenzepine also is in a pharmaceutical composition in an amount, in pirenzepine dihydrochloride, of from 25 mg to 600 mg, to be administered at a daily dose of from 25 mg to 600 mg, normally from 150 mg to 600 mg (in pirenzepine dihydrochloride).

The above donepezil amounts in the above composition and donepezil daily doses include low amounts and daily doses administered to patients suffering from a Hypocholinergic Disorder such as dementia of Alzheimer type during to the initial titration period. After this initial titration period, donepezil or pharmaceutically acceptable salts thereof may continue to be titrated upward from daily doses equivalent to from 23.01 mg up to 80 mg or from 25 mg to 80 mg of donepezil hydrochloride. Normally, said donepezil dose (daily or per unit form), is equivalent to from 25 mg to 60 mg or from 25 mg to 40 mg of donepezil hydrochloride.

The present invention also provides a pharmaceutical combination comprising an M1-antagonist selected from the group consisting of pirenzepine and pharmaceutically acceptable salts and solvates thereof, at a dose (daily or per unit form) equivalent to from 75 mg to 300 mg of pirenzepine dihydrochloride; and donepezil or a pharmaceutically acceptable salt thereof, at a dose (daily or per unit form) equivalent to from 23.01 mg to 80 mg or from 25 mg to 80 mg of donepezil hydrochloride.

This combination allows a safe treatment of patients suffering from a Hypocholinergic Disorder such as Alzheimer's disease by its administration to said patient.

In particular, for this treatment, pirenzepine or a pharmaceutically acceptable salt or solvate thereof is administered twice or three times per day at a daily dose (in pirenzepine dihydrochloride) of from 150 mg to 600 mg and donepezil or a pharmaceutically acceptable salt thereof is administered once a day at the dose (in donepezil hydrochloride) of from 23.01 to 80 mg or from 25 mg to 80 mg.

Normally, said donepezil dose is equivalent to from 25 mg to 60 mg or from 25 mg to 40 mg of donepezil hydrochloride.

To this purpose, pirenzepine and donepezil are each formulated in a pharmaceutical composition comprising pirenzepine or a pharmaceutically acceptable salt or solvate thereof, in an amount (in pirenzepine dihydrochloride) of from 25 mg to 600 mg in admixture with a pharmaceutical carrier or vehicle; and, respectively, donepezil or a pharmaceutically acceptable salt thereof, in an amount (in donepezil hydrochloride) of from 5 mg to 80 mg, in admixture with a pharmaceutical carrier.

Preferably, donepezil or pharmaceutically acceptable salt thereof is present, in said respective composition, in an amount equivalent to from 23.01 mg to 80 mg or from 25 mg to 80 mg of donepezil hydrochloride.

Said combination includes fixed-dose combinations and kits, as illustrated in “The formulations” section below.

In said combination, including fixed-dose combinations, each of the respective compositions normally is in dosage unit form and the above amounts of pirenzepine and donepezil are per unit form.

In particular, a pharmaceutical composition in dosage unit form comprising pirenzepine or a pharmaceutically acceptable salt or solvate thereof, in an amount per unit form equivalent to from 25 mg to 600 mg of pirenzepine dihydrochloride is adapted to be administrable to a patient a daily dose (in pirenzepine dihydrochloride) of from 50 mg to 600 mg, in combination, including fixed-dose combinations, with a pharmaceutical composition in dosage unit form comprising donepezil or a pharmaceutically acceptable salt thereof, in an amount per unit form equivalent to from 5 mg to 80 mg of donepezil hydrochloride, also adapted to be administrable to said patient at a daily dose (in donepezil hydrochloride) of from 5 mg to 80 mg.

Thus, in combination with a selective peripheral M1 receptor antagonist such as preferably, pirenzepine, in an amount per unit form (in pirenzepine dihydrochloride) of from 50 mg to 600 mg, from 100 mg to 600, from 150 mg to 600 mg or from 150 mg to 500 mg and at a daily dose (in pirenzepine dihydrochloride) of from 50 mg to 600 mg, from 100 mg to 600, from 150 mg to 600 mg or from 150 mg to 500 mg, preferably from 25 mg to 300 mg or from 75 mg to 300 mg, donepezil may be administered in an amount per unit form (in donepezil hydrochloride) of from 5 mg to 80 mg or from 10.01 mg to 60 mg, normally from 12.5 mg to 60 mg, from 12.5 mg to 50 mg, from 12.5 mg to 40 mg or from 25 mg to 30 mg and administered at a daily dose (in donepezil hydrochloride) of from 5 mg to 60 mg, normally from 10.01 mg to 60 mg or from 25 mg to 60 mg, advantageously from 12.5 mg to 60 mg, from 12.5 mg to 50 mg, from 12.5 mg to 40 mg or from 25 mg to 30 mg, preferably from 25 mg to 80 mg.

Preferably, in combination with the selective peripheral M1 receptor antagonist such as pirenzepine, in an amount per unit form (in pirenzepine dihydrochloride) of from 75 mg to 300 mg and at a daily dose (in pirenzepine dihydrochloride) of from 75 mg to 300 mg or from 150 mg to 600 mg, donepezil may be administered in an amount per unit form and at a daily dose equivalent to from 23.01 mg to 80 mg, or from 25 mg to 80 mg normally from 25 mg to 60 mg or from 25 mg to 40 mg of donepezil hydrochloride.

Thus, according to a preferred embodiment, the invention provides a pharmaceutical composition comprising donepezil or a pharmaceutically acceptable salt thereof, in an amount equivalent to from 23.01 mg to 80 mg of donepezil hydrochloride, in admixture with a pharmaceutical carrier or vehicle.

In particular, in said composition, said donepezil or a pharmaceutically acceptable salt thereof is present in an amount equivalent to from 25 mg to 80 mg of donepezil hydrochloride

Normally, according to this preferred embodiment, this pharmaceutical composition is in dosage unit form and the above amount ranges are per unit form.

Preferably, said pirenzepine/donepezil combination is a fixed dose combination in admixture with a pharmaceutical carrier.

To this purpose, the invention provides a pharmaceutical composition comprising pirenzepine or a pharmaceutical acceptable salt or solvate thereof, in an amount equivalent to from 25 mg to 600 mg of pirenzepine dihydrochloride; and donepezil or a pharmaceutical acceptable salt thereof, in an amount equivalent to from 5 mg to 80 mg of donepezil hydrochloride, in admixture with a pharmaceutical carrier.

Normally, this pharmaceutical composition is in dosage unit form and the above amounts (or amount ranges) are per dosage unit form.

Advantageously, in this composition, pirenzepine or a pharmaceutical acceptable salt or solvate thereof is present in an amount equivalent to from 75 mg to 300 mg of pirenzepine dihydrochloride; and donepezil or a pharmaceutical acceptable salt thereof is present in an amount equivalent to from 25 mg to 80 mg of donepezil hydrochloride.

Preferably, this composition is in dosage unit form and said amount of pirenzepine or a pharmaceutical acceptable salt or solvate thereof and, respectively, of donepezil or a pharmaceutical acceptable salt thereof are per unit form.

### First Aspect of the Invention

A first aspect of the present invention provides a method for increasing the maximal tolerated dose of donepezil in a patient suffering from a Hypocholinergic Disorder such as Alzheimer's Disease, Lewy Body Dementia, Parkinson's disease dementia, or Mild Cognitive Impairment without concurrent, appreciable adverse effects, which comprises administering to said patient in need thereof, donepezil in combination with pirenzepine, whereby an enhanced acetyl choline esterase inhibition in the CNS of said patient is achieved and the symptoms of a Hypocholinergic Disorder such as Alzheimer's Disease, Lewy Body Dementia, Parkinson's disease dementia, or Mild Cognitive Impairment in said patient are improved.

In particular, according to this first aspect the invention provides a method for the treatment of a Hypocholinergic Disorder such as dementia of the Alzheimer-type, which comprises administering to a patient in need of said treatment pirenzepine or a pharmaceutically acceptable salt or solvate thereof, in combination with a donepezil or pharmaceutically acceptable salt thereof daily dose equivalent to from 5 mg to 80 mg of donepezil hydrochloride.

Normally, these donepezil or pharmaceutically acceptable salt thereof daily dose are equivalent to from 5 mg to 60 mg or from 5 mg to 40 mg of donepezil hydrochloride.

These daily doses include doses used in the initial titration period.

Preferably, in combination with pirenzepine, said donepezil daily dose is equivalent to from 25 mg to 80 mg of donepezil hydrochloride.

Further, according to this first aspect, the invention provides a method for the treatment of a Hypocholinergic Disorder such as Alzheimer's Disease, Lewy Body Dementia, Parkinson's disease dementia, or Mild Cognitive Impairment which comprises administering to a patient in need of said treatment, pirenzepine or a pharmaceutically acceptable salt or solvate thereof, in combination with donepezil or a pharmaceutically acceptable salt thereof daily dose equivalent to from 15 mg to 80 mg of donepezil hydrochloride.

According to an embodiment of this first aspect, the invention provides a method for the treatment of a Hypocholinergic Disorder such as dementia of Alzheimer type, which comprises administering to a patient in need of said treatment, pirenzepine or a pharmaceutically acceptable salt or solvate thereof, at a daily dose of from 75 mg to 600 mg or from 75 mg to 300 mg (in pirenzepine dihydrochloride), in combination with donepezil or pharmaceutically acceptable salt thereof, at a daily dose equivalent to from 23.01 mg to 80 mg.

In particular, according to this embodiment, pirenzepine or a pharmaceutically acceptable salt or solvate thereof, at a daily dose equivalent to from 75 mg to 300 mg of pirenzepine dihydrochloride, is administered to said patient in combination with a donepezil daily dose equivalent to from 25 mg to 80 mg of donepezil hydrochloride.

Said donepezil daily dose is normally equivalent to from 25 mg to 60 mg or from 25 mg to 40 mg of donepezil hydrochloride.

To this purpose, pirenzepine and donepezil are each formulated in a pharmaceutical composition comprising pirenzepine, in an amount (in pirenzepine dihydrochloride) of from 25 mg to 600 mg in admixture with a pharmaceutical carrier; and, respectively, donepezil, in an amount (in donepezil hydrochloride) of from 5 mg to 80 mg, in admixture with a pharmaceutical carrier or vehicle.

Normally, in said respective composition, said donepezil is present in an amount equivalent to from 23.01 mg to 80 mg or from 25 mg to 80 mg of donepezil hydrochloride, in admixture with a pharmaceutical carrier or vehicle.

Preferably, pirenzepine is present in said compositions in an amount equivalent to from 75 mg to 300 mg of pirenzepine dihydrochloride, in admixture with a pharmaceutical carrier or vehicle. Said composition may be in IR-formulation or ER-formulation.

Preferably, donepezil is present in said compositions in an amount equivalent to from 25 mg to 80 mg of donepezil hydrochloride, in admixture with a pharmaceutical carrier.

Normally, each of the above compositions is in dosage unit form and said pirenzepine amounts and, respectively, said donepezil amounts are per unit form.

According to this first aspect, the present invention also provides a method for the treatment of a Hypocholinergic Disorder such as dementia of Alzheimer type, which comprises administering to a patient in need of said treatment, pirenzepine or a pharmaceutically acceptable salt or solvate thereof, in a fixed-dose combination with donepezil or pharmaceutically acceptable salt thereof and a pharmaceutical carrier or vehicle.

In particular, said method comprises administering to a patient in need of said treatment a pharmaceutical composition comprising a pharmaceutical carrier and a fixed dose combination of pirenzepine and donepezil.

To this purpose, pirenzepine and donepezil are formulated in a pharmaceutical composition comprising pirenzepine, in an amount (in pirenzepine dihydrochloride) of from 25 mg to 600 mg; and donepezil, in an amount (in donepezil hydrochloride) of from 5 mg to 80 mg, in admixture with a pharmaceutical carrier or vehicle. P Normally, in said composition, said donepezil is present in an amount equivalent to from 23.01 mg to 80 mg or from 25 mg to 80 mg of donepezil hydrochloride.

Preferably, in said composition, pirenzepine is present in an amount equivalent to from 75 mg to 300 mg of pirenzepine dihydrochloride, and donepezil is present in an amount equivalent to from 25 mg to 80 mg of donepezil hydrochloride, in admixture with a pharmaceutical carrier or vehicle.

Normally, the above compositions are in dosage unit form and said pirenzepine and donepezil amounts are per unit form.

### Second Aspect of the Invention

According to a second aspect, the invention provides for the use of a selective, peripheral anticholinergic agent (sPAChA), preferably pirenzepine, for the preparation of a medicament for the treatment of a Hypocholinergic Disorder, such as Alzheimer's Disease, Lewy Body Dementia, Parkinson's disease dementia, and Mild Cognitive Impairment, in combination with donepezil.

The efficacy of pirenzepine in reducing, preventing, or treating the symptoms of a Hypocholinergic Disorder such as Alzheimer's Disease, Lewy Body Dementia, Parkinson's disease dementia, and Mild Cognitive Impairment in a patient is due to the fact that pirenzepine allows for a safe increase in the therapeutic daily doses of donepezil up to 80 mg. For instance, an increase in the maximal tolerated dose of donepezil is able to maximally alleviate disease-associated dementia.

Thus, a higher degree of acetyl choline esterase inhibition in the CNS of said patient is achieved and the symptoms of said Hypocholinergic Disorder are improved to a greater extent without concurrent, appreciable adverse effects.

In particular, according to this second aspect, the invention concerns the use of pirenzepine or a pharmaceutically acceptable salt or solvate thereof for the preparation of a medicament comprising said pirenzepine or pharmaceutically acceptable salt or solvate thereof, at a dose (or amount) equivalent to from 25 mg to 600 mg of pirenzepine dihydrochloride, for the treatment of a Hypocholinergic Disorder such as a dementia of the Alzheimer-type in combination with donepezil or a pharmaceutically acceptable salt thereof, at a dose equivalent to from 5 mg to 80 mg of donepezil hydrochloride. Normally, said dose (in donepezil hydrochloride) is from 5 mg to 60 mg or from 5 mg to 40 mg of donepezil hydrochloride.

These daily doses include doses used in the initial titration period.

In particular, in combination with pirenzepine, donepezil or a pharmaceutically acceptable salt thereof may be administered at a daily dose equivalent to from 10.01 mg to 80 mg, from 10.01 mg to 60 mg, from 12.5 mg to 60 mg, from 12.5 mg to 50 mg, from 12.5 mg to 40 mg or from 25 mg to 30 mg of donepezil hydrochloride.

Advantageously, said donepezil daily dose, in combination with pirenzepine, is equivalent to from 15 mg to 80 mg of donepezil hydrochloride.

More advantageously, said donepezil daily dose, in combination with pirenzepine, is equivalent to from 23.01 mg to 80 mg.

Preferably, said donepezil daily dose is equivalent to from 25 mg to 80 mg of donepezil hydrochloride.

This aspect of the invention also concerns the use of pirenzepine or a pharmaceutically acceptable salt or solvate thereof for the preparation of a medicament comprising said pirenzepine or pharmaceutically acceptable salt or solvate thereof at a dose (or amount) equivalent to from 25 mg to 600 mg of pirenzepine dihydrochloride, for the treatment of a Hypocholinergic Disorder, such as Alzheimer's Disease, Lewy Body Dementia, Parkinson's disease dementia, or Mild Cognitive Impairment, in combination with donepezil or a pharmaceutically acceptable salt thereof at a daily dose equivalent to from 15 mg to 80 mg of donepezil hydrochloride.

According to an embodiment, said donepezil daily dose is equivalent to a range selected from the group consisting of from 12.5 mg to 60 mg, from 12.5 mg to 50 mg, from 12.5 mg to 40 mg, from 5 mg to 20 mg, from 25 mg to 60 mg and from 25 mg to 30 mg of donepezil hydrochloride.

To this purpose, said pirenzepine and said donepezil are each formulated in a pharmaceutical composition comprising pirenzepine, in an amount (in pirenzepine dihydrochloride) of from 25 mg to 600 mg in admixture with a pharmaceutical carrier; and, respectively, donepezil, in an amount (in donepezil hydrochloride) of from 5 mg to 80 mg, in admixture with a pharmaceutical carrier or vehicle.

The above donepezil amount-range includes low doses adapted to be administered to a patient during the initial titration period of treatment.

Normally, in said respective composition, said donepezil is present in an amount equivalent to from 15 mg to 80 mg, normally from 23.01 mg to 80 mg or from 25 mg to 80 mg of donepezil hydrochloride, in admixture with a pharmaceutical carrier or vehicle.

Pirenzepine is present in an amount that reduces peripherally mediated adverse effects that would be caused by the administration of a dose of donepezil sufficient to maximally alleviate disease-associated dementia.

In particular, said medicament is a pharmaceutical composition comprising pirenzepine or a pharmaceutically acceptable salt or solvate thereof, in an amount per unit form of from 25 mg to 300 mg, from 50 mg to 600 mg, from 100 mg to 600 mg, from 150 mg to 600 mg and from 150 mg to 500 mg of pirenzepine dihydrochloride.

Preferably, said composition comprises pirenzepine in an amount equivalent to from 75 mg to 300 mg of pirenzepine dihydrochloride, in admixture with a pharmaceutical carrier or vehicle. Said composition may be in IR-formulation or ER-formulation.

Preferably, donepezil is present in said composition in an amount equivalent to from 25 mg to 80 mg of donepezil hydrochloride, in admixture with a pharmaceutical carrier.

Normally, each of the above compositions is in dosage unit form and said pirenzepine amounts and, respectively, said donepezil amounts are per unit form.

According to an advantageous embodiment, said medicament is a pharmaceutical composition comprising said pirenzepine or pharmaceutically acceptable salt or solvate thereof in an amount equivalent to from 75 mg to 300 mg of pirenzepine dihydrochloride. This medicament is for the treatment of a Hypocholinergic Disorder such as a dementia of the Alzheimer-type in combination with donepezil or a pharmaceutically acceptable salt thereof at a daily dose equivalent to from 15 mg to 80 mg of donepezil hydrochloride. Preferably, in combination with pirenzepine said donepezil daily dose is from 23.01 mg to 80 mg, from 25 mg to 80 mg, from 25 mg to 60 mg or from 25 mg to 40 mg of donepezil hydrochloride.

In particular, the present invention provides the use of pirenzepine for the preparation of a medicament consisting of a pharmaceutical composition comprising pirenzepine or a pharmaceutically acceptable salt or solvate thereof, in an amount equivalent to from 25 mg to 600 mg of pirenzepine dihydrochloride, in admixture with a pharmaceutical carrier or vehicle, for the treatment of a Hypocholinergic Disorder in combination with donepezil or a pharmaceutically acceptable salt thereof, also in a pharmaceutical composition comprising donepezil or a pharmaceutically acceptable salt or solvate thereof in an amount equivalent to from 5 mg to 80 mg, in admixture with a pharmaceutical carrier or vehicle.

Normally, in said composition, said donepezil is present in an amount equivalent to from 23.01 mg to 80 mg or from 25 mg to 80 mg of donepezil of donepezil hydrochloride.

Preferably, said pirenzepine in said composition is present in an amount (in pirenzepine dihydrochloride) of from 75 mg to 300 mg and said donepezil in said composition is present in an amount (in donepezil hydrochloride) of from 25 mg to 80 mg.

According to this second aspect, the present invention also provides the use of pirenzepine for the preparation of a medicament for the treatment of a Hypocholinergic Disorder such as dementia of Alzheimer type, said medicament consisting of a pharmaceutical composition comprising a pharmaceutical carrier or vehicle and a fixed-dose combination of pirenzepine or a pharmaceutically acceptable salt or solvate thereof and donepezil or a pharmaceutically acceptable salt thereof.

In said fixed-dose combination, pirenzepine is present at a dose of from 25 mg to 600 mg (in pirenzepine dihydrochloride), and donepezil is present at a dose of from 5 mg to 80 mg (in donepezil hydrochloride).

According to an embodiment, in said fixed-dose combination pirenzepine is present at a dose (in pirenzepine dihydrochloride) of from 25 mg to 300 mg, and donepezil is present at a dose (in donepezil hydrochloride) of from 23.01 mg to 80 mg.

According to a preferred embodiment, said pirenzepine is present at a dose of from 75 mg to 300 mg (in pirenzepine dihydrochloride), and donepezil is present at a dose of from 25 mg to 80 mg (in donepezil hydrochloride).

In particular, said medicament is a pharmaceutical composition comprising said pirenzepine, in an amount equivalent to from 75 mg to 300 mg of pirenzepine dihydrochloride, and said donepezil, in an amount equivalent to from 25 mg to 80 mg of donepezil hydrochloride, in admixture with a pharmaceutical carrier or vehicle.

The compositions described in this section are normally in dosage unit form and the above pirenzepine and, respectively, donepezil amounts or doses are per unit form.

### Third Aspect of the Invention

According to a third aspect, the present invention provides a selective, peripheral anticholinergic agent (sPAChA), preferably pirenzepine or a pharmaceutically acceptable salt or solvate thereof, for use for the treatment of a Hypocholinergic Disorder such as a dementia of the Alzheimer-type in combination with a donepezil or a pharmaceutical acceptable salt thereof, whereby the maximal tolerated dose of donepezil is increased, so that a higher degree of acetyl choline esterase inhibition in the CNS is achieved and the symptoms of a Hypocholinergic Disorder such as a dementia of Alzheimer type are improved to a greater extent without concurrent, appreciable adverse effects.

The efficacy of pirenzepine in preventing, reducing, or treating the symptoms of a Hypocholinergic Disorder such as a dementia of Alzheimer type is due to the fact that pirenzepine allows for a safe increase in the therapeutic daily doses of donepezil up to 80 mg. This increase in the dose of donepezil is able to maximally alleviate disease-associated dementia.

Thus, according to this third aspect, the invention provides pirenzepine or a pharmaceutically acceptable salt or solvate thereof for use for the treatment of a Hypocholinergic Disorder in combination with a donepezil or pharmaceutical acceptable salt thereof daily dose equivalent to from 5 mg to 80 mg of donepezil hydrochloride.

Said donepezil dose is normally equivalent to from 5 mg to 60 mg or from 5 mg to 40 mg, of donepezil hydrochloride.

These daily doses include doses used in the initial titration period.

In particular, in combination with pirenzepine, donepezil or a pharmaceutically acceptable salt thereof is administered at a daily dose equivalent to from 10.01 mg to 80 mg, from 10.01 mg to 60 mg, from 12.5 mg to 60 mg, from 12.5 mg to 50 mg, from 12.5 mg to 40 mg or from 25 mg to 30 mg of donepezil hydrochloride.

Advantageously, said donepezil daily dose, in combination with pirenzepine, is equivalent to from 15 mg to 80 mg of donepezil hydrochloride.

More advantageously, said donepezil daily dose, in combination with pirenzepine, is equivalent to from 23.01 mg to 80 mg.

Preferably said donepezil daily dose, in combination with pirenzepine, is equivalent to from 25 mg to 80 mg of donepezil hydrochloride.

This third aspect also provides pirenzepine or a pharmaceutically acceptable salt or solvate thereof for use for the treatment of a Hypocholinergic Disorder, such as Alzheimer's Disease, Lewy Body Dementia, Parkinson's disease dementia, or Mild Cognitive Impairment, in combination with a donepezil or a pharmaceutical acceptable salt thereof daily dose equivalent to from 23 mg to 138 mg of donepezil hydrochloride.

In particular, according to this aspect, the present invention provides pirenzepine or pharmaceutically acceptable salt or solvate thereof, at a dose (or an amount) equivalent to from 25 mg to 600 mg of pirenzepine dihydrochloride, for use in the treatment of cognitive impairment caused by a Hypocholinergic Disorder in combination donepezil or a pharmaceutically acceptable salt thereof daily dose equivalent to from 5 mg to 80 mg of donepezil hydrochloride.

These daily doses include doses used in the initial titration period.

In addition, in combination with pirenzepine, donepezil or a pharmaceutically acceptable salt thereof is administered at a daily dose equivalent to a range selected from the group consisting of from 10.01 mg to 60 mg, from 15 mg to 80 mg, from 12.5 mg to 60 mg, from 12.5 mg to 50 mg, from 12.5 mg to 40 mg, from 5 mg to 20 mg, from 25 mg to 60 mg and from 25 mg to 30 mg of donepezil hydrochloride.

To this purpose, pirenzepine is in a pharmaceutical composition for enabling a safe higher acetyl choline esterase inhibition in the CNS of a patient suffering from a Hypocholinergic Disorder, such as a dementia of the Alzheimer type, said patient taking a dose of donepezil higher than the maximal tolerated dose or a dose of donepezil sufficient to maximally alleviate disease-associated dementia, comprising, as an active ingredient, pirenzepine in admixture with a pharmaceutical carrier. By such an enablement of higher acetylcholine esterase inhibition, not otherwise attainable when donepezil is taken alone, the symptoms of a Hypocholinergic Disorder, such as dementia of the Alzheimer type, in said patients are thus further improved.

In particular, pirenzepine and donepezil are each formulated in a pharmaceutical composition comprising pirenzepine, in an amount (in pirenzepine dihydrochloride) of from 25 mg to 600 mg in admixture with a pharmaceutical carrier; and, respectively, donepezil, in an amount (in donepezil hydrochloride) of from 5 mg to 80 mg, in admixture with a pharmaceutical carrier or vehicle.

The above donepezil amount-range includes low doses to be administered to a patient during the initial titration period of treatment.

Normally, in said respective composition, said donepezil is present in an amount equivalent to from 15 mg to 80 mg, normally from 23.01 mg to 80 mg or from 25 mg to 80 mg of donepezil hydrochloride, in admixture with a pharmaceutical carrier or vehicle.

For the use described herein, pirenzepine is formulated in pharmaceutical compositions comprising, as an active ingredient thereof, said pirenzepine, in the aforementioned amount per unit form equivalent to from 25 mg to 600 mg pf pirenzepine hydrochloride, in admixture with a pharmaceutical carrier or vehicle.

Advantageously, these pharmaceutical compositions comprise pirenzepine in an amount (in pirenzepine dihydrochloride) of from 25 mg to 300 mg.

Preferably, these compositions comprise pirenzepine in an amount equivalent to from 75 mg to 300 mg of pirenzepine dihydrochloride, in admixture with a pharmaceutical carrier or vehicle.

Said composition may be in IR-formulation or ER-formulation.

Preferably, donepezil is present in said composition in an amount equivalent to from 25 mg to 80 mg of donepezil hydrochloride, in admixture with a pharmaceutical carrier.

Normally, each of the above compositions is in dosage unit form and said pirenzepine amounts and, respectively, said donepezil amounts are per unit form.

The compositions comprising pirenzepine according to the present invention allow the administration of 1.1 up to 6-8 times the maximal tolerated dose of donepezil (administered alone) to patients suffering of a Hypocholinergic Disorder such as Alzheimer Disease without clinically significant symptoms of peripheral cholinergic system overstimulation.

The compositions are preferably formulated in dosage unit forms for oral or parenteral, in particular transdermal, administration, wherein each of the active ingredients is mixed with a pharmaceutical carrier or vehicle.

The pharmaceutical compositions prepared using pirenzepine according to the present invention are indicated in the treatment of the symptoms of a Hypocholinergic Disorder such as a dementia of the Alzheimer-type, in order to improve to a greater extent said symptoms by allowing an increase of the currently used doses of donepezil, concurrently or sequentially administered therewith, without the dose-limiting side-effects that would hinder said increase of said therapeutic doses.

Pirenzepine, for use in combination with donepezil, is adapted to be administered in a pharmaceutical composition comprising an amount per unit form (in pirenzepine dihydrochloride) of from 50 mg to 600 mg, from 100 mg to 600 mg, from 150 mg to 600 mg or from 150 mg to 500 mg, at a daily dose (in pirenzepine dihydrochloride) of from 50 mg to 600 mg, from 100 mg to 600 mg, from 150 mg to 600 mg or from 150 mg to 500 mg.

Thanks to the protective action of pirenzepine, donepezil may be administered in an amount per unit form and at a daily dose (in donepezil hydrochloride) in a range selected from the group consisting of from 5 mg to 80 mg, from 5 mg to 60 mg, from 10.01 mg to 60 mg, from 12.5 mg to 60 mg, from 12.5 mg to 50 mg, from 12.5 mg to 40 mg and from 25 mg to 30 mg.

Donepezil may also be advantageously administered in an amount per unit form and at a daily dose (in donepezil hydrochloride) of from 15 mg to 80 mg.

Donepezil is preferably administered in an amount per unit form and at a daily dose (in donepezil hydrochloride) of 25 mg to 80 mg, normally from 25 mg to 60 mg or from 25 mg to 40 mg.

For this administration, donepezil or a pharmaceutically acceptable salt thereof is formulated in a pharmaceutical composition comprising said donepezil or a pharmaceutically acceptable salt thereof, in an amount equivalent to from 5 mg to 80 mg of donepezil hydrochloride, in admixture with a pharmaceutical carrier.

Alternatively, donepezil or a pharmaceutically acceptable salt thereof is formulated in a pharmaceutical composition comprising said donepezil or a pharmaceutically acceptable salt thereof in an amount equivalent to from 15 mg to 80 mg of donepezil hydrochloride, in admixture with a pharmaceutical carrier.

Donepezil or a pharmaceutically acceptable salt thereof may also be formulated in a pharmaceutical composition comprising said donepezil or a pharmaceutically acceptable salt thereof in an amount equivalent to from 25 mg to 80 mg of donepezil hydrochloride, in admixture with a pharmaceutical carrier.

Preferred pharmaceutical compositions for oral administration using pirenzepine as preferred active ingredient may contain from 20 to 300 mg, advantageously from 25 mg to 300 mg, preferably from 50 to 150 mg (in pirenzepine dihydrochloride), of said active ingredient in IR formulations or from 40 mg to 600 mg or from 75 mg to 300 mg, preferably from 100 mg to 600 mg, in ER formulations. Said preferred pharmaceutical compositions allow the concurrent or sequential administration of a donepezil daily dose equivalent to from 15 mg to 80 mg or from 15 mg to 100 mg of donepezil hydrochloride, to a patient suffering from a Hypocholinergic Disorder such as a dementia of Alzheimer type.

In particular, according to this third aspect, the present invention provides pirenzepine or a pharmaceutically acceptable salt or solvate thereof, in a pharmaceutical composition comprising said pirenzepine or a pharmaceutically acceptable salt or solvate thereof in an amount equivalent to from 75 mg to 300 mg of pirenzepine dihydrochloride, in admixture with a pharmaceutical carrier, for use for the treatment of a Hypocholinergic Disorder such as a dementia of the Alzheimer type, in combination with a donepezil or a pharmaceutical acceptable salt thereof, also in a pharmaceutical composition comprising said donepezil or pharmaceutical acceptable salt thereof in an amount equivalent to from 25 mg to 80 mg, of donepezil hydrochloride, in admixture with a pharmaceutical carrier.

Said composition comprising said pirenzepine or a pharmaceutically acceptable salt or solvate thereof in an amount equivalent to from 75 mg to 300 mg of pirenzepine dihydrochloride, in admixture with a pharmaceutical carrier may be in an IR-formulation or in an ER-formulation.

For this use, donepezil is normally present, in said pharmaceutical composition, in an amount equivalent to from 25 mg to 80 mg, from 25 mg to 60 mg or from 25 mg to 40 mg, of donepezil hydrochloride.

Normally, these pharmaceutical compositions comprising from 75 mg to 300 mg of pirenzepine and, respectively, from 25 mg to 80 mg, from 25 mg to 60 mg or from 25 mg to 40 mg of donepezil are in dosage unit form, and these amounts are per unit form.

Finally, according to another embodiment of this third aspect, the invention provides a pharmaceutical composition for use in the treatment of a Hypocholinergic disorder such as dementia of Alzheimer type, comprising a pharmaceutical carrier and a fixed-dose combination of pirenzepine or a pharmaceutically acceptable salt or solvate thereof, in an amount equivalent to from 75 mg to 300 mg of pirenzepine dihydrochloride and donepezil or a pharmaceutically acceptable salt thereof, in an amount equivalent to from 25 mg to 80 mg of pirenzepine dihydrochloride.

In said fixed-dose combination, donepezil may be present in an amount of from 25 mg to 60 mg or from 25 mg to 40 mg.

The compositions according to this section are normally in dosage unit form and the above pirenzepine and, respectively, donepezil amounts are per unit form.

### Fourth Aspect of the Invention

A fourth aspect of the present invention provides a pharmaceutical combination for treatment of a Hypocholinergic Disorder such as a dementia of the Alzheimer-type comprising pirenzepine and donepezil.

In particular, this fourth aspect of the invention provides an anti-Alzheimer pharmaceutical combination comprising pirenzepine or a pharmaceutically acceptable salt or solvate thereof, and an effective daily dose of donepezil or a pharmaceutically acceptable salt thereof.

According to this fourth aspect of the present invention, said pharmaceutical combination comprises pirenzepine or a pharmaceutically acceptable salt or solvate thereof, at a daily dose equivalent to from 25 mg to 600 mg of pirenzepine dihydrochloride; and donepezil or a pharmaceutically acceptable salt thereof, at a daily dose equivalent to from 5 mg to 80 mg of donepezil hydrochloride.

In particular, in combination with a pirenzepine or a pharmaceutically acceptable salt thereof daily dose equivalent to from 75 mg to 600 mg of pirenzepine dihydrochloride, donepezil or a pharmaceutically acceptable salt thereof is administered at a daily dose equivalent to from 10.01 mg to 80 mg, from 10.01 mg to 60 mg, from 12.5 mg to 60 mg, from 12.5 mg to 50 mg, from 12.5 mg to 40 mg or from 25 mg to 30 mg of donepezil hydrochloride.

Advantageously, said donepezil daily dose, in combination with pirenzepine, is equivalent to from 15 mg to 80 mg of donepezil hydrochloride.

More advantageously, said donepezil daily dose, in combination with pirenzepine, is equivalent to from 23.01 mg to 80 mg of donepezil hydrochloride.

Preferably, said donepezil daily dose, in combination with pirenzepine, is equivalent to from 25 mg to 80 mg of donepezil hydrochloride.

These daily doses include doses used in the initial titration period.

According to a preferred embodiment of this fourth aspect of the invention, said pirenzepine or pharmaceutically acceptable salt or solvate thereof is administered at a daily dose equivalent to from 75 mg to 300 mg of pirenzepine dihydrochloride; and said donepezil or pharmaceutical acceptable salt thereof is administered at a daily dose equivalent to from 25 mg to 80 mg of donepezil hydrochloride.

According to this fourth aspect, pirenzepine or a pharmaceutically acceptable salt or solvate thereof and donepezil or a pharmaceutically acceptable salt thereof are each formulated in a pharmaceutical composition, each in admixture with a pharmaceutical carrier or vehicle.

To this purpose, pirenzepine and donepezil are each formulated in a pharmaceutical composition comprising pirenzepine, in an amount (in pirenzepine dihydrochloride) of from 25 mg to 600 mg in admixture with a pharmaceutical carrier; and, respectively, donepezil, in an amount (in donepezil hydrochloride) of from 5 mg to 80 mg, in admixture with a pharmaceutical carrier or vehicle.

The above donepezil amount-range includes low doses adapted to be administered to a patient during the initial titration period of treatment.

According to an embodiment, in said respective composition,
said pirenzepine is preferably present in an amount equivalent to from 75 mg to 600 mg of pirenzepine dihydrochloride; and
said donepezil is preferably present in an amount equivalent to from 15 mg to 80 mg of donepezil hydrochloride.

Advantageously, said donepezil is present in said composition in an amount equivalent to from 23.01 mg to 80 mg of donepezil hydrochloride.

Preferably, said donepezil is present in said composition in an amount equivalent to from 25 mg to 80 mg of donepezil hydrochloride.

Preferably, each of said pirenzepine and donepezil compositions is in dosage unit form, and the above pirenzepine and donepezil amounts are per unit form.

In the combination according to this fourth aspect of the invention, the pirenzepine or a pharmaceutically acceptable salt or solvate thereof component is present in a pharmaceutical composition comprising pirenzepine or a pharmaceutically acceptable salt or solvate thereof in an amount per unit form of from 25 mg to 600 mg of pirenzepine dihydrochloride. This composition is adapted to be used at a daily dose (in pirenzepine dihydrochloride) of from 25 mg to 600 mg, from 50 mg to 600 mg, from 100 mg to 600, from 150 mg to 600 mg or from 150 mg to 500 mg in the combination with donepezil for the treatment of Hypocholinergic Disorder such as Alzheimer's Disease, Lewy Body Dementia, Parkinson's disease dementia, or Mild Cognitive Impairment.

In the combination according to this fourth aspect of the invention, the donepezil or pharmaceutically acceptable salt thereof component is present in a pharmaceutical composition comprising donepezil or a pharmaceutically acceptable salt thereof in an amount per unit form equivalent to from 5 mg to 80 mg of donepezil hydrochloride, from 5 mg to 60 mg of donepezil hydrochloride, from 15 mg to 80 mg of donepezil hydrochloride, from 23 mg to 80 mg, of donepezil hydrochloride, from 23.01 mg to 80 mg of donepezil hydrochloride or from 25 mg to 80 mg of donepezil hydrochloride. Normally, during the treatment of a Hypocholinergic disorder such as a dementia of Alzheimer type, said pharmaceutical composition will comprise donepezil or a pharmaceutically acceptable salt thereof in an amount per unit form equivalent to a range selected from the group consisting of from 10.01 mg to 60 mg, from 15 mg to 80 mg, from 23.01 mg to 80 mg, from 12.5 mg to 60 mg, from 12.5 mg to 50 mg, from 12.5 mg to 40 mg, from 5 mg to 20 mg, from 25 mg to 30 mg, from 25 mg to 60 mg and from 25 mg to 80 mg of donepezil hydrochloride.

According to an advantageous embodiment, in this combination, pirenzepine is present in a pharmaceutical composition comprising pirenzepine or a pharmaceutically acceptable salt or solvate thereof in an amount per unit form equivalent to from 75 mg to 300 mg of pirenzepine dihydrochloride. It is for use in, or adapted to, said treatment of a Hypocholinergic disorder such as Alzheimer-type dementia, at a daily dose (in pirenzepine dihydrochloride), of from 75 mg to 300 mg, in combination with a donepezil daily dose equivalent to from 25 mg to 80 mg, normally from 25 mg to 60 mg or from 25 mg to 40 mg of donepezil hydrochloride.

According to a preferred embodiment of this fourth aspect, said combination for the treatment of a Hypocholinergic Disorder, in particular Alzheimer Disease, comprises
pirenzepine or a pharmaceutically acceptable salt or solvate thereof, formulated in a pharmaceutical composition in an amount per unit form equivalent to from 75 mg to 300 mg of pirenzepine dihydrochloride, in admixture with a pharmaceutical carrier or vehicle, in IR-formulation or ER-formulation; and, respectively,
donepezil or a pharmaceutically acceptable salt thereof, formulated in a pharmaceutical composition in an amount per unit form equivalent to from 25 mg to 80 mg of donepezil hydrochloride, in admixture with a pharmaceutical carrier or vehicle.

In this combination, pirenzepine, in said formulation may be administered at a daily dose equivalent to from 75 mg to 600 mg, normally from 75 mg to 300 mg of pirenzepine dihydrochloride; and donepezil, in said formulation, is preferably administered at a daily dose equivalent to from 25 mg to 80 mg, normally from 25 mg to 60 mg or 25 mg to 40 mg of donepezil hydrochloride.

Advantageously, the pharmaceutical compositions, prepared by using pirenzepine according to these four aspects of the present invention, are in unit forms also containing other active ingredients, in particular donepezil which acts as a cholinergic agent in the CNS to improve the symptoms of a Hypocholinergic Disorder, such as Alzheimer disease, or dementia of Alzheimer type, in a quantity sufficient to maximally alleviate disease-associated neurobehavioral symptoms, with a minimum of treatment-associated adverse effects.

### Fifth Aspect of the Invention

A fifth aspect of the present invention provides a pharmaceutical composition for, or adapted to, the treatment of a Hypocholinergic Disorder, comprising a pharmaceutical carrier and a fixed-dose combination of pirenzepine or a pharmaceutically acceptable salt or solvate thereof and donepezil or a pharmaceutically acceptable salt thereof. Normally, said composition is an anti-Alzheimer composition.

Preferably, said fixed-dose combination is in an unit form comprising said pirenzepine or a pharmaceutically acceptable salt or solvate thereof in an amount equivalent to from 25 mg to 600 mg of pirenzepine dihydrochloride, and said donepezil or a pharmaceutically acceptable salt thereof, in an amount equivalent to from 5 mg to 80 mg of donepezil hydrochloride.

Said Hypocholinergic Disorder may be Alzheimer Disease, Lewy Body Dementia, Parkinson's disease dementia, or Mild Cognitive impairment.

Alternatively, said donepezil may be present in said fixed-dose combination, in an amount equivalent to a range selected from the group consisting of from 5 mg to 60 mg, from 20 mg to 60 mg, from 25 mg to 60 mg, from 15 mg to 80 mg, from 20 mg to 80 mg, and from 25 mg to 80 mg of donepezil hydrochloride.

According to an embodiment, in said fixed-dose combination,
said pirenzepine or a pharmaceutically acceptable salt or solvate thereof is present in an amount equivalent to from 50 mg to 600 mg, from 100 mg to 600, from 150 mg to 600 mg or from 150 mg to 500 mg of pirenzepine dihydrochloride; and
said donepezil or a pharmaceutically acceptable salt thereof is present in an amount equivalent to from 10.01 mg to 60 mg, from 12.5 mg to 60 mg, from 12.5 mg to 50 mg, from 12.5 mg to 40 mg or from 25 mg to 30 mg of donepezil hydrochloride.

According to another embodiment, in said fixed-dose combination,
said pirenzepine or a pharmaceutically acceptable salt or solvate thereof is present in an amount equivalent to from 50 mg to 600 mg, from 100 mg to 600, from 150 mg to 600 mg or from 150 mg to 500 mg of pirenzepine dihydrochloride; and
said donepezil or a pharmaceutically acceptable salt thereof is present in an amount equivalent to from 15 mg to 80 mg of donepezil hydrochloride.

According to a further embodiment, in said fixed-dose combination, said pirenzepine or a pharmaceutically acceptable salt or solvate thereof is present in an amount equivalent to from 25 mg to 300 mg of pirenzepine dihydrochloride; and said donepezil or a pharmaceutically acceptable salt thereof is present in an amount equivalent to from 15 mg to 80 mg of donepezil hydrochloride.

Advantageously, in said fixed-dose combination, said pirenzepine or a pharmaceutically acceptable salt or solvate thereof is present in an amount equivalent to from 75 mg to 300 mg; and said donepezil or a pharmaceutically acceptable salt thereof is present in an amount equivalent to from 25 mg to 80 mg of donepezil hydrochloride.

Alternatively, in said fixed-dose combination, donepezil or a pharmaceutically acceptable salt thereof is normally present in an amount equivalent to from 25 mg to 60 mg or from 25 mg to 40 mg of donepezil hydrochloride.

For its anti-Alzheimer use or prescription, the fixed-dose combination of this section is formulated in a pharmaceutical composition, preferably in dosage unit form. In said respective compositions, the above amounts of pirenzepine and, respectively, of donepezil are per unit form.

### Sixth Aspect of the Invention

According to a sixth aspect, the present invention provides a pharmaceutical composition to improve the treatment of a human Hypocholinergic disorder such as a dementia of Alzheimer-type, which comprises a pharmaceutical carrier and a mixture of pirenzepine and of donepezil wherein
donepezil is present in a quantity sufficient to maximally and safely improve (normally by reducing, preventing or treating) the symptoms of said Hypocholinergic Disorder such as dementia of Alzheimer-type, and
pirenzepine, which does not appreciably penetrate the blood brain barrier, is present in a second quantity that reduces peripherally mediated adverse effects that would be caused by donepezil if administered without pirenzepine.

In particular, according to this sixth aspect, the present invention provides a pharmaceutical composition comprising
(a) pirenzepine or a pharmaceutically acceptable salt or solvate thereof, in an amount equivalent to from 25 mg to 600 mg of pirenzepine dihydrochloride; and
(b) donepezil or a pharmaceutically acceptable salt thereof, in an amount equivalent to from 5 mg to 80 mg of donepezil hydrochloride;
in admixture with at least a pharmaceutical carrier or vehicle.

Alternatively, in this aspect, the present invention provides a pharmaceutical composition comprising
(a) pirenzepine or a pharmaceutically acceptable salt or solvate thereof, in an amount equivalent to from 25 mg to 600 mg of pirenzepine dihydrochloride; and
(b) donepezil or a pharmaceutically acceptable salt thereof, in an amount equivalent to from 15 mg to 80 mg of donepezil hydrochloride;
in admixture with at least a pharmaceutical carrier or vehicle.

According to this sixth aspect, the present invention also provides a pharmaceutical composition comprising
(a) pirenzepine or a pharmaceutically acceptable salt or solvate thereof, in an amount equivalent to from 25 mg to 600 mg of pirenzepine dihydrochloride; and
(b) donepezil or a pharmaceutically acceptable salt thereof, in an amount equivalent to from 5 mg to 60 mg of donepezil hydrochloride;
in admixture with at least a pharmaceutical carrier or vehicle.

For its use in (or prescription for) the treatment of a Hypocholinergic disorder, the pharmaceutical composition of this sixth aspect of the present invention is formulated in an unit form suitable for the administration to a patient.

In the composition of the present invention, for immediate release or extended release, pirenzepine is present in an amount per unit form (in pirenzepine dihydrochloride) of from 20 mg to 600 mg, normally from 25 mg to 600 mg in an IR- or ER formulation; and donepezil is present in an amount of 100% to 800% or from 100% to 600% of the amount of donepezil contained in the currently administered IR dosage unit forms.

According to the present invention, pirenzepine is present, in an IR unit form, in an amount (in pirenzepine dihydrochloride) ranging from 25 mg to 300 mg or from 75 mg to 300 mg.

In an ER unit form, pirenzepine is present in an amount (in pirenzepine dihydrochloride) ranging from 50 mg to 600 mg, normally from 75 mg to 600 mg, from 100 mg to 500 mg, or from 100 mg to 300 mg, preferably from 75 mg to 300 mg.

Normally, in a pharmaceutical composition in dosage unit form, pirenzepine is present in an amount per IR-unit form equivalent to from 50 mg to 300 mg of pirenzepine dihydrochloride, or in an amount per ER-form equivalent to from 50 mg to 600 mg of pirenzepine dihydrochloride.

Preferably, in a pharmaceutical composition in dosage unit form, pirenzepine or a pharmaceutically acceptable salt thereof is present in an amount per unit form equivalent to from 75 mg to 300 mg of pirenzepine dihydrochloride, in an IR- or ER-unit form.

In unit form for immediate release or extended release, donepezil is present in an amount of from about 100% to about 600% of the amount of donepezil contained in the currently administered IR dosage unit forms for the treatment of a Hypocholinergic Disorder such as a dementia of the Alzheimer-type.

More particularly, donepezil is present in an IR unit form, in an amount ranging from 100% to 800%, from 100% to 600%, from 100% to 400%, preferably from 150% to 800%, from 150% to 600% or from 150% to 400%, of the amount of donepezil contained in the currently administered IR dosage unit forms for the palliative treatment of a Hypocholinergic Disorder such as dementia of the Alzheimer-type or, in an ER unit form, in an amount ranging from 150% to 600%, preferably from 200% to 600% of the amount of donepezil contained in the currently administered unit dosage IR forms for the treatment of a Hypocholinergic Disorder such as dementia of the Alzheimer-type. For example, donepezil is present in an amount (in donepezil hydrochloride) of from 5 mg to 80 mg, from 5 mg to 60 mg, preferably from 7.5 mg to 60 mg, per dosage unit. In another example, donepezil is present in an amount (in donepezil hydrochloride) of from 15 mg to 100 mg, from 15 mg to 80 mg or from 34.5 mg to 80 mg, per dosage unit.

According to a first embodiment of this sixth aspect, the invention provides a pharmaceutical composition comprising
(a) pirenzepine or a pharmaceutically acceptable salt or solvate thereof, in an amount equivalent to from 25 mg to 300 mg of pirenzepine dihydrochloride; and
(b) donepezil or a pharmaceutically acceptable salt thereof, in an amount equivalent to from 5 mg to 80 mg of donepezil hydrochloride;
in admixture with a pharmaceutical carrier or vehicle.

According to a second embodiment of this sixth aspect, the invention provides a pharmaceutical composition comprising
(a) pirenzepine or a pharmaceutically acceptable salt or solvate thereof, in an amount equivalent to from 75 mg to 300 mg of pirenzepine dihydrochloride; and
(b) donepezil or a pharmaceutically acceptable salt thereof, in an amount equivalent to from 25 mg to 80 mg of donepezil hydrochloride;
in admixture with a pharmaceutical carrier or vehicle.

According to a third embodiment of this sixth aspect, the invention provides a pharmaceutical composition comprising
(a) pirenzepine or a pharmaceutically acceptable salt or solvate thereof, in an amount equivalent to from 150 mg to 300 mg of pirenzepine dihydrochloride; and
(b) donepezil or a pharmaceutically acceptable salt thereof, in an amount equivalent to from 25 mg to 80 mg of donepezil hydrochloride;
in admixture with a pharmaceutical carrier or vehicle.

Alternatively, according to this third embodiment of this third embodiment of this sixth aspect, this pharmaceutical composition comprises pirenzepine or a pharmaceutically acceptable salt or solvate thereof in an amount per unit form equivalent to from 150 mg to 300 mg of pirenzepine dihydrochloride; and donepezil or a pharmaceutically acceptable salt thereof, in an amount per unit form equivalent to from 25 mg to 80 mg of donepezil hydrochloride, in admixture with a pharmaceutical carrier or vehicle.

### The Formulations

The pharmaceutical compositions of the present invention are formulated with the classic excipients suitable for different ways of administration. Particularly advantageous are the formulations in the form of tablets, multi-score tablets, coated tables, orally disintegrating tablets (orodispersible tablets), extended-release tablets, hard or soft capsules, extended-release capsules, patches for transdermal administration, liquid oral solutions, syrups or suspensions in a predetermined unit form, and vials for the intravenous or subcutaneous administration.

The pharmaceutical compositions may be formulated in oral forms such as tablets or gelatin capsules, wherein pirenzepine or donepezil; or both the active ingredients, are in admixture with a carrier or vehicle. Said carrier or vehicle may include a diluent, such as cellulose, dextrose, lactose, mannitol, sorbitol or sucrose; a lubricant, such as, acid, calcium or magnesium stearate, polyethylene glycol, silica, or talc; and if needed, a binder such as magnesium aluminum silicate, gelatin, methylcellulose, sodium carboxymethylcellulose, or polyvinylpyrrolidone.

Donepezil orodispersible tablets may comprise a taste masking agent, such as polacrilin potassium that limits or suppress the release of donepezil in the oral cavity of the patient, as disclosed in US 2011/0060008.

Said oral forms may be tablets coated with sucrose or with various polymers, including polishing natural products such as beeswax.

Alternatively, the tablets can be manufactured by using carriers such as acrylic and methacrylic acid polymers and copolymers; cellulose derivatives such as hydroxypropylethylcellulose; or other appropriate materials. These materials confer a prolonged or delayed activity by progressively releasing a predetermined quantity of pirenzepine (or pharmaceutically acceptable salt or solvate thereof) or donepezil (or pharmaceutically acceptable salt thereof).

The oral formulations can also be in form of capsules allowing the extended release of pirenzepine (or a pharmaceutically acceptable salt or solvate thereof); of donepezil (or pharmaceutically acceptable salt thereof); or of both the active ingredients.

A fixed-dose combination according to the present invention may be a dosage unit form consisting of a capsule comprising for example pirenzepine dihydrochloride monohydrate, in an amount of 75 mg or 150 mg (in pirenzepine dihydrochloride); and donepezil hydrochloride, in an amount of 15 mg or 30 mg, in admixture with a pharmaceutical carrier.

For the intended use in the treatment of a Hypocholinergic Disorder such as a dementia of the Alzheimer type, in combination with donepezil, pirenzepine is formulated in a pharmaceutical composition, wherein said pirenzepine is in admixture with a pharmaceutical carrier. For said treatment, also donepezil is formulated in a pharmaceutical composition, wherein said donepezil is in admixture with a pharmaceutical carrier.

The dosage, i.e. the amount of active ingredient in a single dose (amount per unit form) to be administered to the patient, can vary widely depending on the age, weight, and the health condition of the patient. This dosage includes the administration of a pirenzepine amount from 25 mg to 600 mg, from 25 mg to 300 mg or from 75 mg to 300 mg, according to the potency the age of the patient, an effective donepezil amount equivalent to from 5 mg to 80 mg or from 5 mg to 60 mg of donepezil hydrochloride or an effective donepezil amount equivalent to from 23 mg to 70 mg or from 25 mg to 80 mg, normally from 25 mg to 60 mg, of donepezil hydrochloride, according to the age of the patient, once a day, according to the strength of the doses of the each of the active ingredient.

The above pharmaceutical compositions are formulated in admixture with a pharmaceutical carrier or vehicle for any administration route. For example, said pharmaceutical compositions are in a pharmaceutical dosage unit form for oral, intranasal, transdermal, or rectal administration.

These unit forms are manufactured according to conventional technologies. Particularly advantageous are the formulations in the form of tablets, multi-score tablets, multi-layer tablets, coated tables, orally disintegrating tablets, extended release tablets, hard or soft capsules, multi-compartment capsules (normally two-compartment capsules), extended-release capsules, suppositories for rectal administration, patches for transdermal administration, liquid oral solutions, syrups or suspensions in a predetermined unit form, apparatus for intravenous infusion, and vials for the intravenous or subcutaneous administration.

The pharmaceutical compositions may be formulated in oral unit forms such as tablets or gelatin capsules wherein each of the pirenzepine, donepezil, or pirenzepine/donepezil fixed-dose combination active ingredients are in admixture with a carrier or vehicle that may include a diluent, such as cellulose, microcrystalline cellulose, dextrose, lactose, mannitol, sorbitol or sucrose; a lubricant, such as, acid, calcium or magnesium stearate, polyethylene glycol, silica, or talc; and if needed, a binder such as magnesium aluminum silicate, gelatin, methylcellulose, sodium carboxymethylcellulose, or polyvinylpyrrolidone.

Said oral unit forms may be tablets coated with sucrose or with various polymers for an immediate release. Alternatively, the tablets can be manufactured by using carriers such as acrylic and methacrylic acid polymers and copolymers; cellulose derivatives such as hydroxypropylethylcellulose; or other appropriate materials having a prolonged or delayed activity by progressively releasing a predetermined quantity of active ingredient. For example, the unit forms may be formulated in tablets in which pirenzepine is in ER-formulation, for example in admixture with hydroxypropyl methyl cellulose or in a film-coated microgranule. Carriers and vehicles for ER-tablets include retardant materials such as acrylic and methacrylic acid polymers and copolymers; the aforementioned cellulose derivatives such as hydroxypropylmethylcellulose, hydroxyethylcellulose, hydroxypropylethylcellulose, hydroxypropylcellulose, methylcellulose, ethylcellulose, or sodium carboxymethylcellulose; gums; waxes; glycerides or aliphatic alcohols or a mixture thereof.

Syrups and orally dispersible tablets may also comprise sweeteners, lubricants, taste-masking agents, binders, and coloring agents.

Unit forms may be formulated in tablets in which Component (a) and Component (b) each in ER-formulation, for example pirenzepine dihydrochloride and donepezil hydrochloride, each in admixture with hydroxypropyl methyl cellulose or in a film-coated microgranule. Donepezil may also be formulated in matrix-type tablets comprising said donepezil in admixture with a pharmaceutical carrier including a hydrophilic polymer, for example as described in U.S. Pat. No. 8,481,565, for a sustained release. These unit forms (a) and (b) are destined to be concurrently or sequentially administered to a patient suffering from a Hypocholinergic Disorder, such as Alzheimer disease or a dementia of the Alzheimer-type, in combination with an oral unit form such as a tablet or gelatin capsule wherein Component (a) is formulated with a diluent and a lubricant in an IR-formulation, or in a tablet or capsule for extended release.

Pirenzepine may be formulated in a pharmaceutical composition, wherein said pirenzepine is in admixture with a pharmaceutical carrier or vehicle. Donepezil may also be formulated in a pharmaceutical composition, wherein said donepezil is in admixture with a pharmaceutical carrier or vehicle.

In the case of separate (concurrent or sequential) administration of said pirenzepine, in an effective amount per unit form, and of said donepezil, in an effective amount per unit form, each of them can be packaged in a kit comprising said pirenzepine, in admixture with a pharmaceutical carrier or vehicle, in a container; and donepezil, in admixture with a pharmaceutical carrier or vehicle, in another, separate container.

In the above combinations, including fixed-dose combinations, of the present invention, the dose of pirenzepine or pharmaceutically acceptable salt or solvate thereof, in pirenzepine dihydrochloride, per unit form, is from 25 mg to 600 mg, in an IR- or ER-formulation, normally in an amount of from 25 mg to 300 mg, preferably from 75 mg to 300 mg, in IR-formulation, or in an amount of from 75 mg to 300 mg, from 100 mg to 600 mg, preferably from 150 mg to 600 mg or from 150 mg to 500 mg. in ER-formulation.

According to another embodiment, the compositions of the present invention are formulated by mixing pirenzepine with a pharmaceutical carrier for extended release in tablets (Tablet A), and donepezil, separately, with a pharmaceutical carrier for an immediate release in tablets (Tablet B) and introducing Tablet A and Tablet B in a capsule for oral administration as described for example in GB 1204580 or in US 2007/0224259 (the contents of which are incorporated herein in their entirety for reference). An advantageous composition according to this embodiment consists of soft or hard gelatin capsules each containing Tablet A comprising pirenzepine dihydrochloride monohydrate, in an amount equivalent to from 25 to 250 mg of pirenzepine dihydrochloride, in admixture with a pharmaceutical carrier in ER formulation; and Tablet B, comprising from 5 to 30 mg of donepezil (as hydrochloride), with a pharmaceutical carrier in an IR-formulation said composition being destined to be administered once per day.

The compositions of the present invention may also be formulated by mixing for example pirenzepine dihydrochloride monohydrate with a pharmaceutical carrier consisting of one or more high molecular fatty acid esters such as ethyl oleate, ethyl linoleate, triolein or gefamate, as described in JPH01149729A.

A preferred unit form according to this embodiment consists of soft or hard gelatin capsules each containing:
Tablet A comprising pirenzepine dihydrochloride monohydrate, in an amount (in pirenzepine dihydrochloride) of from 75 mg to 300 mg, from 150 mg to 600 mg or from 150 mg to 500 mg, in admixture with a pharmaceutical carrier in ER formulation; and
Tablet B, comprising from 25 mg to 80 mg, normally from 25 to 60 mg, of donepezil hydrochloride, with a pharmaceutical carrier in an 1R-formulation. Said unit form is destined to be administered once per day.

According to a further embodiment, a composition according to the present invention is formulated in a bi-layer tablet, each layer releasing the drug contained therein, without any reciprocal interference, as described for example in WO 2006/089493 (the contents of which are incorporated herein in their entirety by reference).

An advantageous composition according to this embodiment consists of
Layer A, comprising pirenzepine dihydrochloride monohydrate, in an amount equivalent to from 75 mg to 300 mg of pirenzepine dihydrochloride, with a pharmaceutical carrier in an ER formulation and
Layer B, comprising donepezil hydrochloride, in an amount of from 5 mg to 80 mg or from 5 to 60 mg, normally from 10.01 to 60 mg or from 25 mg to 30 mg, in admixture with a pharmaceutical carrier in an IR-formulation, said composition being destined to be administered once per day.

In addition, the compositions according to the present invention may be also formulated in a tri-layer tablet, as described for example in EP 2848261 (the contents of which are incorporated herein in their entirety by reference). In this tablet, one of the external layer, in IR-formulation, releasing pirenzepine, the central layer, in ER-formulation for a sustained release of pirenzepine and the other external layer, in IR-formulation or ER-formulation, for the release of donepezil doses.

An advantageous tri-layer tablet according to this embodiment consists of
Layer A, as a composition comprising pirenzepine dihydrochloride monohydrate, in an amount equivalent to 25 mg of pirenzepine dihydrochloride, in admixture with a pharmaceutical carrier in IR-formulation;
Layer B (central layer), as a composition comprising pirenzepine dihydrochloride monohydrate, in an amount equivalent to 50 mg of pirenzepine dihydrochloride, in admixture with a pharmaceutical carrier in an ER formulation; and
Layer C, as a composition comprising from 23.01 mg to 40 mg, normally from 25 mg to 30 mg, of donepezil hydrochloride an IR-formulation,
said composition being destined to be administered once per day.

A further advantageous tri-layer tablet comprises
Layer A, as a composition comprising pirenzepine dihydrochloride monohydrate, in an amount equivalent to from 25 mg to 300 mg of pirenzepine dihydrochloride, in admixture with a pharmaceutical carrier;
Layer B (central layer), as a composition comprising placebo (inert material) only; and
Layer C, as a composition comprising from 5 mg to 80 mg of donepezil hydrochloride, in admixture with a pharmaceutical carrier,

Preferably, in this further advantageous trilayer tablet,
the composition of Layer A comprises pirenzepine dihydrochloride monohydrate, in an amount equivalent to from 75 mg to 300 mg of pirenzepine dihydrochloride, in admixture with a pharmaceutical carrier; and
the composition of Layer C, comprises from 25 mg to 80 mg of donepezil hydrochloride, in admixture with a pharmaceutical carrier.

According to another embodiment, the compositions of the present invention are formulated in oral disintegrable tablets. Particularly advantageous compositions according to this embodiment are orally disintegrable tablets comprising
pirenzepine dihydrochloride monohydrate, in an amount equivalent to from 50 to 250 mg of pirenzepine dihydrochloride; and
from 5 to 50 mg, normally from 12.5 mg to 40 mg, of donepezil hydrochloride;
in admixture with a pharmaceutical carrier in an IR-formulation for buccal mucosa absorption, said composition being destined to be administered once or twice per day.

A typical 75-mg pirenzepine tablet contains 78.18 mg of pirenzepine dihydrochloride monohydrate, corresponding to 75 mg of pirenzepine dihydrochloride, 18 mg of microcrystalline cellulose (Avicel PH 101), 9.6 mg of maize starch, 6 mg of polyvinylpyrrolidone, 0.2 mg of hydroxpropylcellulose, 3.7 mg of talc, 1.5 mg of magnesium stearate, 3.7 mg of ultra-amylopectin, and microcrystalline cellulose (Avicel PH 102) to reach a total of 123 mg.

A typical 100-mg pirenzepine tablet contains 104.24 mg of pirenzepine dihydrochloride monohydrate, corresponding to 100 mg of pirenzepine dihydrochloride, 24 mg of microcrystalline cellulose (Avicel PH 101), 12.8 mg of maize starch, 8 mg of polyvinylpyrrolidone, 0.3 mg of hydroxpropylcellulose, 4.9 mg of talc, 2 mg of magnesium stearate, 4.9 mg of ultra-amylopectin, and microcrystalline cellulose (Avicel PH 102) to reach a total of 164 mg.

A typical 300-mg pirenzepine tablet contains 312.73 mg of pirenzepine dihydrochloride monohydrate, corresponding to 300 mg of pirenzepine dihydrochloride, 72 mg of microcrystalline cellulose (Avicel PH 101), 38.5 mg of maize starch, 24 mg of polyvinylpyrrolidone, 0.7 mg of hydroxpropylcellulose, 14.7 mg of talc, 5.9 mg of magnesium stearate, 14.7 mg of ultra-amylopectin, and microcrystalline cellulose (Avicel PH 102) to reach a total of 490 mg.

The therapeutic efficacy is measured by the degree to which cognitive and other neurobehavioral disabilities associated with dementias of the Alzheimer-type, as documented by the use of standard scales, are reduced.

### Kits

The present invention also provides a kit or package containing a medicament comprising a pharmaceutical combination, or a pharmaceutical composition as described herein, accompanied by instructions for use of the same in the treatment of a Hypocholinergic Disorder by simultaneous, sequential or separate administration of the preparations to a patient in need thereof. The kit form is particularly advantageous when pirenzepine and donepezil must be administered in different dosage forms or are administered at different dosage intervals.

In one embodiment, a kit or package combining two separate units comprises Unit Form Component (a), wherein pirenzepine, normally in an amount per unit form equivalent to from 25 mg to 300 mg or from 75 mg to 300 mg of pirenzepine dihydrochloride, in admixture with a pharmaceutical carrier in IR-formulation; and instructions for use of the same for treatment of a Hypocholinergic Disorder in a patient in need thereof, in combination with donepezil.

In another embodiment, a kit of the present invention is kit comprising pharmaceutical composition (a) comprising pirenzepine, normally in an amount equivalent to from 75 mg to 600 mg or from 75 mg to 300 mg of pirenzepine dihydrochloride in admixture with a pharmaceutical carrier in a ER-formulation; and a pharmaceutical composition (b) comprising donepezil, in an amount per unit form equivalent to from 15 mg to 80 mg, normally from 25 mg to 80 mg of donepezil hydrochloride, in IR-formulation; and instructions for use of the same for treatment of a Hypocholinergic Disorder by simultaneous, sequential or separate administration of the preparations to a patient in need thereof in a patient in need thereof.

In a further embodiment, the kit of the present invention is a kit comprising pharmaceutical composition (a) comprising pirenzepine or a pharmaceutically acceptable salt or solvate thereof, normally in an amount equivalent to from 75 mg to 300 mg of pirenzepine dihydrochloride in admixture with a pharmaceutical carrier in a ER-formulation; and a pharmaceutical composition (b) comprising donepezil or a pharmaceutically acceptable salt thereof, in an amount per unit form equivalent to a range selected from the group consisting of from 23.1 mg to 80 mg, from 25 mg to 80 mg and from 25 mg to 60 mg of donepezil hydrochloride in admixture with a pharmaceutical carrier, in an IR-formulation or in an ER-formulation; and instructions for use of the same for treatment of a Hypocholinergic Disorder by simultaneous, sequential or separate administration of the preparations to a patient in need thereof.

According to a specific embodiment, in the pharmaceutically composition included in the above kits, said pirenzepine or pharmaceutically acceptable salt or solvate thereof is pirenzepine dihydrochloride monohydrate, said donepezil or pharmaceutically acceptable salt or solvate thereof is donepezil hydrochloride, and said Hypocholinergic disorder is dementia of Alzheimer type.

The following examples illustrate the invention.

### EXAMPLE 1

The ability of pirenzepine to prevent the GI adverse effects of donepezil in humans was tested.

A Phase I study was conducted in human subjects receiving a single oral dose of donepezil hydrochloride (herein below “donepezil”) with or without a single oral dose of pirenzepine dihydrochloride monohydrate (herein below “pirenzepine”). The study was a single center, single-blind study.

The objective of the study was to demonstrate that pirenzepine could safely attenuate the GI adverse effects of donepezil hydrochloride (herein below “donepezil”). given in single doses ranging from 5 to 50 mg.

To be enrolled in the study, participants had to meet the following inclusion/exclusion criteria:

### Key Inclusion Criteria

1. Healthy male or female volunteers between the ages of 18 and 50 years inclusive.
2. Females of childbearing potential were required to agree to be abstinent or else use any two of the following medically acceptable forms of contraception from the Screening Period through 14 days after the study Exit Visit: hormonal contraception, condom with spermicidal jelly, diaphragm or cervical cap with spermicidal jelly, or IUD. A female whose male partner had had a vasectomy was required to agree to use one additional form of medically acceptable contraception. Subjects were required agree to practice the above birth control methods for 14 days after the final visit as a safety precaution.
3. Females of non-childbearing potential, defined as surgically sterile (status post hysterectomy, bilateral oophorectomy, or bilateral tubal ligation) or post-menopausal for at least 12 months, did not require contraception during the study. Post-menopausal had to be confirmed by a serum FSH test at Screening and the reason must be documented in the source documents.
4. Subjects had to be in good health as determined by their medical history including personal and family psychiatric history, physical examination, ECG, vital signs, and laboratory tests. A subject with a medical abnormality could be included only if the investigator or designee considered that the abnormality would not introduce significant additional risk to the subject's health or interfere with study objectives.
5. Subjects had to have a body mass index between 19 and 30 kg/m² (both inclusive).
6. Subjects were required to have signed an informed consent form indicating that they understood the purpose of and procedures required for the study and were willing to participate in the study and comply with the study procedures and restrictions.
7. Subjects had to be able to swallow multiple pills simultaneously.

### Key Exclusion Criteria:

The criteria for exclusion of a subject from enrollment in the study were as follows:
1. Any clinically relevant acute or chronic diseases, which could interfere with the subjects' safety during the trial, expose them to undue risk, or interfere with the study objectives.
2. History or presence of gastrointestinal, hepatic, or renal disease or other condition known to interfere with the absorption, distribution, metabolism or excretion of drugs.
3. History of substance abuse, known drug addiction, or positive test for drugs of abuse or alcohol.
4. Current use of potent CYP 3A4 inhibitors
5. History of drug or other significant allergy.
6. Treatment with centrally active drugs or those affecting peripheral cholinergic transmission within 3 months of study entry.
7. Current or Former Smokers (except subjects who stopped smoking 1 year or more before enrollment in the Study) including tobacco products.
8. Excessive daily consumption of xanthines containing drinks.
9. Subjects unwilling to curtail prolonged intensive physical exercise during the study conduct (from the Screening visit until the last dose of study drug).
10. Positive test result for hepatitis B surface antigen, hepatitis C antibody.
11. Positive test result for HIV 1 and 2 serology.
12. Use of any prescription or over-the-counter medication within 14 days prior to admission on Day-1. In addition, any medications with central effects are prohibited for a period equal to 5 times the drug half-life prior to admission (Day-1), should this period be longer than 14 days.
13. Subjects unlikely to co-operate during the study, and/or be questionably compliant in the opinion of the investigator.
14. Subjects unable to be contacted in case of an emergency.
15. Intake of an investigational drug within 30 days of study entry.

Following enrollment in the study, participants received single increasing oral doses of donepezil given once daily in the morning. The starting dose of donepezil was 5 mg and the dose was increased daily or every other day by 5 mg increments up to the maximum protocol-allowed dose of 60 mg or up to the highest tolerated dose whichever comes first. Once a subject had reached a dose of 60 mg of donepezil or his/her first intolerable dose (“FID-1”) whichever came first, upward dose escalation was discontinued. FID was defined as:
One (1) episode of vomiting, or
Two (2) episodes of retching, or
One (1) episode of severe nausea (Grade 3; defined as nausea interfering with activities of daily living or inadequate oral caloric or fluid intake; tube feeding, total parenteral nutrition or hospitalization indicated) lasting more than 1 hour, or
Three (3) consecutive episodes at every 4 hours ratings of moderate nausea (Grade 2; defined as subjectively symptomatic, but not interfering with activities of daily living), or
One (1) episode of moderate diarrhea (Grade 2; defined as 4-6 stools more than at baseline).

When a subject reached FID-1 on donepezil alone, the subject was washed out for 7 days, and then received single daily doses of donepezil starting at 5 mg and titrated upward by 5 mg increments, together with an oral dose of pirenzepine, until subjects again reached an intolerable dose (FID-2). The dose of pirenzepine was initially 100 mg (in pirenzepine dihydrochloride) together with donepezil. When a subject reached FID-2, he/she received on the following day, the same dose of donepezil with a dose of 200 mg of pirenzepine (in pirenzepine dihydrochloride). If the dose-limiting side effects of FID-2 were not antagonized, the subject received on the next day the same dose of donepezil with a higher 300 mg dose of pirenzepine (in pirenzepine dihydrochloride). Once the dose-limiting side effect of FID-2 were antagonized, dose escalation of donepezil was resumed using the dose of pirenzepine that antagonized the FID-2 side effects until the subject again reached an intolerable dose (FID-3).

On each study day, subjects were followed up for up to 8 hours following drug administration for AEs, vital signs, ECGs. In addition, a laboratory panel was taken at screening and at the end of the study.

All subjects reached FID-1 (donepezil alone) during the study. The dose limiting toxicity was mainly vomiting in all subjects. Concomitant administration of pirenzepine with donepezil prevented the occurrence of GI adverse events thus enabling the safe increase in the dose of donepezil.

Taken together, results showed that the co-administration of pirenzepine with donepezil attenuated GI AEs reported with donepezil alone and enabled the safe increase of the dose of donepezil.

In conclusion, pirenzepine enables the safe administration to a human being of a dose of donepezil otherwise not tolerated when administering donepezil alone.

### EXAMPLE 2

Combination immediate release tablets of pirenzepine dihydrochloride monohydrate and donepezil hydrochloride were made. Eight different strengths of donepezil hydrochloride tablets were manufactured. Tablets contained 5, 10, 15, 20, 30, 40, 50, or 60 mg of donepezil hydrochloride associated with either 75, 100, 200, or 300 mg (in pirenzepine dihydrochloride) of pirenzepine dihydrochloride monohydrate. The diversity of strengths of tablets was manufactured to enable dose titration to each patient's “best” dose, defined as a dose at which the subject experience maximum cognitive benefit with acceptable side effects.

### EXAMPLE 3

A pirenzepine-donepezil fixed-dose combination is formulated by wet granulation in tablets comprising the following pharmaceutical composition

| | | |
|---|---|---|
| Pirenzepine dihydrochloride monohydrate | 78.18 | mg |
| Donepezil hydrochloride | 25.00 | mg |
| Lactose monohydrate | 46.00 | mg |
| Maize starch | 24.50 | mg |
| Highly dispersed silicon dioxide | 0.50 | mg |
| Microcrystalline cellulose | 19.62 | mg |
| Hydroxypropylcellulose | 2.50 | mg |
| Magnesium stearate | 0.70 | mg |

The calculated amount of pirenzepine dihydrochloride monohydrate and of donepezil hydrochloride are blended with the calculated parts of lactose monohydrate, a portion of microcrystalline cellulose (19 parts), maize starch, and highly dispersed silicon dioxide The intimately mixed powder is wet granulated using a 4% aqueous solution of the hydroxypropylcellulose and then dried in a drying oven. The dry granulate is blended with the remaining parts (0.62) of microcrystalline cellulose and with the magnesium stearate. The composition thus obtained is compressed and coated with a film including talc and hypromellose, to provide coated tablets weighing 197 mg, containing 78.18 mg of pirenzepine dihydrochloride monohydrate and 25 mg of donepezil hydrochloride.

### EXAMPLE 4

By operating as described in Example 3, a coated tablet weighing 400 mg is prepared having the following composition: pirenzepine dihydrochloride monohydrate. 156.36 mg, corresponding to 150 mg of pirenzepine dihydrochloride, donepezil hydrochloride 60 mg, lactose 93 mg, maize starch 49 mg, microcrystalline cellulose 39.24 mg (38 parts plus 1.24 parts), highly dispersed silicon dioxide 1.00 mg, hydroxypropylcellulose 5.00 mg, and magnesium stearate 1.40 mg.

### EXAMPLE 5

By operating as described in Example 3, a coated tablet weighing 750 mg is prepared having the following composition: pirenzepine dihydrochloride monohydrate 372.72 mg, corresponding to 300 mg of pirenzepine dihydrochloride, donepezil hydrochloride 60 mg, lactose 180 mg, maize starch 96 mg, microcrystalline cellulose 39.24 mg (72 parts plus 2.48 parts), highly dispersed silicon dioxide 10 mg, hydroxypropylcellulose 10.00 mg, and magnesium stearate 2.80 mg.

1. A selective peripheral M1 receptor antagonist selected from the group consisting of pirenzepine and pharmaceutically acceptable salts or solvates thereof for use for the treatment of Hypocholinergic Disorders in combination with a donepezil or a pharmaceutical acceptable salt or solvate thereof daily dose equivalent to from 5 mg to 80 mg of donepezil hydrochloride.

2. The selective peripheral M1 receptor antagonist for use according to claim 1, wherein said donepezil or pharmaceutically acceptable salt thereof is administered at daily dose equivalent to from 5 mg to 60 mg of donepezil hydrochloride.

3. The selective peripheral M1 receptor antagonist for use according to claim 1, wherein said donepezil or pharmaceutically acceptable salt thereof daily dose is equivalent to from 15 mg to 80 mg of donepezil hydrochloride.

4. The selective peripheral M1 receptor antagonist for use according to claim 1, wherein said donepezil or pharmaceutically acceptable salt thereof daily dose is equivalent to from 25 mg to 80 mg of donepezil hydrochloride.

5. The selective peripheral M1 receptor antagonist for use according to claim 1, wherein said pirenzepine and said donepezil are each formulated in a pharmaceutical composition comprising pirenzepine or a pharmaceutically acceptable salt or solvate thereof, in admixture with a pharmaceutical carrier and, respectively, donepezil or a pharmaceutically acceptable salt thereof, in admixture with a pharmaceutical carrier.

6. The selective peripheral M1 receptor antagonist for use according to claim 5 wherein
said pirenzepine or pharmaceutically acceptable salt or solvate thereof is formulated in a pharmaceutical composition in an amount equivalent to from 25 mg to 600 mg of pirenzepine dihydrochloride, in admixture with a pharmaceutical carrier; and, respectively,
said donepezil or a pharmaceutical acceptable salt thereof is also formulated in a pharmaceutical composition in an amount equivalent to from 5 mg to 80 mg of donepezil hydrochloride, in admixture with a pharmaceutical carrier.

7. The selective peripheral M1 receptor antagonist for use according to claim 6, wherein said donepezil or pharmaceutical acceptable salt thereof is present in said composition in an amount equivalent to from 15 mg to 80 mg of donepezil hydrochloride.

8. The selective peripheral M1 receptor antagonist for use according to claim 6, wherein, in said composition, said donepezil or pharmaceutical acceptable salt thereof is present in an amount equivalent to from 23.01 mg to 80 mg of donepezil hydrochloride.

9. The selective peripheral M1 receptor antagonist for use according to claim 5, wherein
said pirenzepine or pharmaceutically acceptable salt or solvate thereof is formulated in a pharmaceutical composition in an amount equivalent to from 75 mg to 300 mg of pirenzepine dihydrochloride, in admixture with a pharmaceutical carrier; and, respectively,
said donepezil or a pharmaceutical acceptable salt thereof is formulated in a pharmaceutical composition in an amount equivalent to from 25 mg to 80 mg of donepezil hydrochloride, in admixture with a pharmaceutical carrier.

10. The selective peripheral M1 receptor antagonist for use according to claim 5, wherein said compositions are in dosage unit form and said amounts of said pirenzepine or pharmaceutical acceptable salt or solvate thereof and of said donepezil or pharmaceutical acceptable salt thereof are per unit form.

11. The selective peripheral M1 receptor antagonist for use according to claim 1, wherein said pirenzepine and said donepezil are both formulated in a pharmaceutical composition comprising a pharmaceutical carrier and a fixed-dose combination of said pirenzepine or a pharmaceutically acceptable salt or solvate thereof, and of said donepezil or a pharmaceutically acceptable salt thereof.

12. (canceled)

13. An anti-hypocholinergic disorder combination comprising
a selective peripheral M1 receptor antagonist selected from the group consisting of pirenzepine and pharmaceutically acceptable salts and solvates thereof and
donepezil or a pharmaceutically acceptable salt thereof.

14. (canceled)

15. A method for the treatment of a hypocholinergic disorder, which comprises administering to a patient in need of said treatment an anti-hypocholinergic disorder combination of claim 13.

16. A pharmaceutical composition comprising
a selective peripheral M1 receptor antagonist selected from the group consisting of pirenzepine and pharmaceutically acceptable salts and solvates thereof; and
donepezil or a pharmaceutically acceptable salt thereof;
in admixture with a pharmaceutical carrier.

17. The pharmaceutical composition according to claim 16, wherein
said pirenzepine or pharmaceutically acceptable salt or solvate thereof is present in an amount equivalent to from 25 mg to 600 mg of pirenzepine dihydrochloride; and
said donepezil or a pharmaceutically acceptable salt thereof is present in an amount equivalent to from 5 mg to 80 mg donepezil hydrochloride.

18. The pharmaceutical composition according to claim 17, wherein said donepezil or a pharmaceutically acceptable salt thereof is present in an amount equivalent to from 15 mg to 80 mg of donepezil hydrochloride.

19. The pharmaceutical composition according to claim 17, wherein said donepezil or a pharmaceutically acceptable salt thereof is present in an amount equivalent to from 23.01 mg to 80 mg of donepezil hydrochloride.

20. The pharmaceutical composition according to claim 17, wherein said pirenzepine or pharmaceutically acceptable salt or solvate thereof is present in an amount equivalent to from 75 mg to 300 mg of pirenzepine dihydrochloride; and said donepezil or pharmaceutically acceptable salt thereof is present in an amount equivalent to from 25 mg to 80 mg of donepezil hydrochloride.

21. The composition according to claim 17, wherein said composition is in dosage unit form and said amounts of said pirenzepine or pharmaceutical acceptable salt or solvate thereof and of said donepezil or pharmaceutical acceptable salt thereof are per unit form.

22. The method for the treatment of a hypocholinergic disorder according to claim 15, wherein the hypocholinergic disorder is Alzheimer disease.

23. The method for the treatment of a hypocholinergic disorder according to claim 15, wherein the hypocholinergic disorder is Lewy body disease, Parkinson's Disease or Mild Cognitive Impairment.
